(19) United States
(12) Patent Application Publication
(10) Pub. No.: US 2011/0137157 A1
(43) Pub. Date: Jun. 9, 2011

(54) IMAGE PROCESSING APPARATUS AND IMAGE PROCESSING METHOD

(75) Inventors: Hiroshi Imamura, Tokyo (JP); Yuta Nakano, Kawasaki-shi (JP); Yoshihiko Iwase, Yokohama-shi (JP); Kiyohide Satoh, Kawasaki-shi (JP); Akihiro Katayama, Zama-shi (JP)

(73) Assignee: CANON KABUSHIKI KAISHA, Tokyo (JP)

(21) Appl. No.: 12/941,351

(22) Filed: Nov. 8, 2010

(30) Foreign Application Priority Data
Dec. 8, 2009 (JP) 2009-278948

(51) Int. Cl. A61B 5/05

(52) U.S. Cl. 600/425

(57) ABSTRACT

This invention concerns the acquisition of diagnosis information data effective for diagnosing eye disease independently of the eye state. An image processing apparatus for processing a tomogram of an eye includes a unit configured to determine an eye feature based on the tomogram and thus determine the eye state, a unit configured to detect, from the tomogram, a detection target to be used to calculate diagnosis information data quantitatively representing the determined eye state, and a unit configured to calculate the diagnosis information data using position information of the detection target. In accordance with the eye state, the detection unit changes the detection target or an algorithm to be used to detect the detection target.

### BACKGROUND OF THE INVENTION

1. Field of the Invention

The present invention relates to an image processing technique for processing a tomogram.

2. Description of the Related Art

Ophthalmic examinations are conventionally made aimed at providing an early diagnosis of lifestyle diseases or high-ranking diseases that lead to blindness. An ophthalmic tomography imaging apparatus such as an OCT (Optical Coherence Tomography) is generally used for the ophthalmic examinations. This is because using the tomographic ophthalmic imaging apparatus such as an OCT makes it possible to observe the internal state of a retinal layer three-dimensionally, and thus render a more reliable diagnosis.

On the other hand, when diagnosing an eye disease (for example, glaucoma, age-rated macular degeneration, or macular edema) using obtained tomograms, it is important to analyze the tomograms and quantitatively extract information effective for diagnosis.

To do this, an image processing apparatus for image analysis and the like are normally connected to the ophthalmic tomography imaging apparatus to enable various kinds of image analysis processing. For example, Japanese Patent Laid-Open No. 2008-073099 discloses a function to detect the boundaries between layers of retina, which are effective for disease diagnosis, from obtained tomograms and outputting them as layer position information.

Note that in this specification, pieces of information that are effective for eye disease diagnosis obtained by analyzing obtained tomograms will generically be referred to as “ophthalmic diagnosis information data” or “diagnosis information data” hereinafter.

However, the function disclosed in Japanese Patent Laid-Open No. 2008-073099 is configured to detect a plurality of boundary positions at once using a predetermined image analysis algorithm so as to simultaneously diagnose a plurality of diseases. For this reason, it may be impossible to appropriately obtain all layer position information depending on the eye state (the presence/absence or type of a disease).

A detailed example will be described. For example, a patient suffering from a disease such as age-rated macular degeneration or macular edema has, in his/her eyes, clumped tissues called achromoderma or white spots generated by lipid in blood accumulated in the retinas. If such a tissue is formed, measurement light is blocked by the tissue upon examination. Hence, the luminance value of a tomogram considerably attenuates in a region deeper than the tissue.

That is, the luminance distribution changes between such a tomogram of an eye and that without the tissues. If the same image analysis algorithm is executed for the region, it may be impossible to obtain effective diagnosis information data. To obtain effective diagnosis information data independently of the eye state, the apparatus is preferably designed to apply an image analysis algorithm suitable for the eye state.

### SUMMARY OF THE INVENTION

The present invention has been made in consideration of the above-described problem. That is, an image processing apparatus for processing a tomogram of an eye, comprising: a determination unit configured to determine a state of a disease in the eye based on information of the tomogram; and a detection unit configured to change, in accordance with the state of the disease in the eye determined by the determination unit, one of a detection target to be used to calculate diagnosis information data quantitatively representing the state of the disease and an algorithm to be used to detect the detection target.

According to the present invention, it is possible to obtain diagnosis information data effective for eye disease diagnosis independently of an eye state.

Further features of the present invention will become apparent from the following description of exemplary embodiments (with reference to the attached drawings).

### BRIEF DESCRIPTION OF THE DRAWINGS

The accompanying drawings, which are incorporated in and constitute a part of the specification, illustrate embodiments of the invention and, together with the description, serve to explain the principles of the invention.

FIGS. 1A and 1B show views explaining the relationship between eye states, eye features, detection targets, and diagnosis information data;

FIG. 2 is a block diagram showing the system configuration of a diagnostic imaging system including an image processing apparatus 201 according to the first embodiment;

FIG. 3 is a block diagram showing the hardware configuration of the image processing apparatus 201;

FIG. 4 is a block diagram showing the functional arrangement of the image processing apparatus 201;

FIG. 5 is a flowchart illustrating the procedure of image analysis processing of the image processing apparatus 201;

FIG. 6 is a flowchart illustrating the procedure of normal eye feature processing of the image processing apparatus 201;

FIG. 7 is a flowchart illustrating the procedure of abnormal eye feature processing of the image processing apparatus 201;

FIG. 8A is a flowchart illustrating the procedure of processing of the image processing apparatus 201 for macular edema;

FIG. 8B is a flowchart illustrating the procedure of processing of the image processing apparatus 201 for age-rated macular degeneration;

FIG. 9 shows views showing examples of weight functions used in an evaluation expression to be used to obtain the normal structure of the retinal pigment epithelium layer boundary;

FIG. 10 is a view showing an example of a wide-angle tomogram including a macular portion and an optic disc portion;

FIG. 11 shows views for explaining the relationship between eye states, eye features, detection targets, and diagnosis information data of the respective parts;

FIG. 12 is a block diagram showing the functional arrangement of an image processing apparatus 1201 according to the second embodiment;

FIG. 13 is a flowchart illustrating the procedure of image analysis processing of the image processing apparatus 1201;

FIGS. 14A and 14B show views for explaining the relationship between eye states, eye features, alignment targets, and follow-up diagnosis information data;

FIG. 15 is a block diagram showing the functional arrangement of an image processing apparatus 1501 according to the third embodiment;

FIG. 16A is a flowchart illustrating the procedure of normal eye feature processing of the image processing apparatus 1501;

FIG. 16B is a flowchart illustrating the procedure of processing of the image processing apparatus 1501 for macular edema; and

FIG. 16C is a flowchart illustrating the procedure of processing of the image processing apparatus 1501 for age-rated macular degeneration.

### DESCRIPTION OF THE EMBODIMENTS

Embodiments of the present invention will be described in detail in accordance with the accompanying drawings.

### First Embodiment

An image processing apparatus according to this embodiment is characterized by diagnosing an eye (disease) state in advance based on information (to be referred to as an “eye feature”) about the shape or presence/absence of a predetermined tissue such as the presence/absence of distortion of the retinal pigment epithelium layer boundary or the presence/absence of a white spot or cyst. The apparatus is also characterized by acquiring diagnosis information data corresponding to an eye state by applying an image analysis algorithm capable of acquiring diagnosis information data corresponding to the diagnosed eye state. The image processing apparatus according to the embodiment will now be described below in detail.

<1. Relationship between Eye States, Eye Features, Detection Targets, and Diagnosis Information Data>

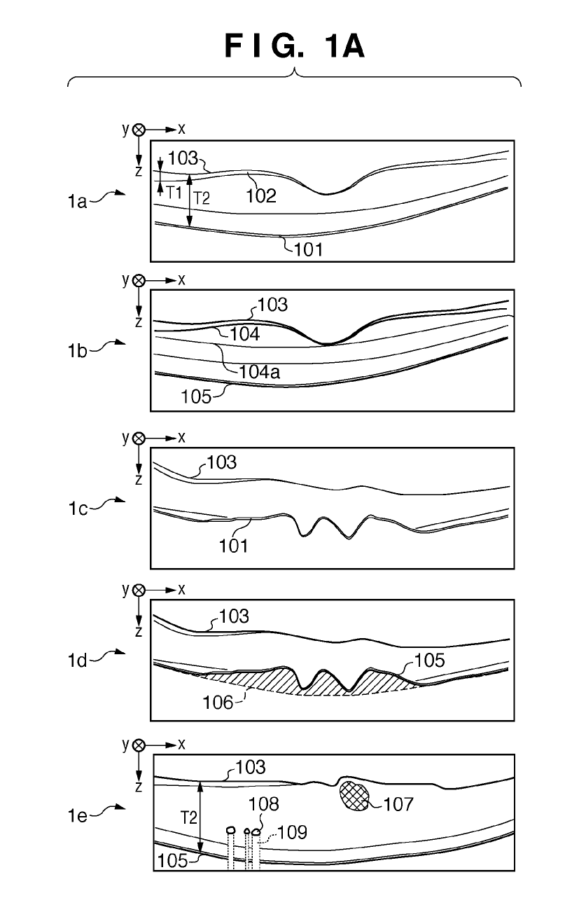

The relationship between eye states, eye features, detection targets, and diagnosis information data will be explained first. In FIG. 1A, 1a to 1e are schematic views showing the tomograms of a macular portion of retina captured by an OCT. In FIG. 1B is a table showing the relationship between eye states, eye features, detection targets, and diagnosis information data. Note that a tomogram of an eye obtained by an OCT is generally a three-dimensional tomogram. Two-dimensional tomograms as part of the three-dimensional tomogram are illustrated here for the descriptive convenience.

Referring to 1a in FIG. 1A, reference numeral 101 denotes a retinal pigment epithelium layer; 102, a nerve fiber layer; and 103, an inner limiting membrane. In the tomogram shown in 1a of FIG. 1A, the presence/absence of a disease such as glaucoma, its degree of progress, recovery condition after treatments, and the like can quantitatively be diagnosed by calculating, for example, the thickness of the nerve fiber layer 102 or the thickness of entire retina (T1 or T2 in 1a of FIG. 1A) as diagnosis information data.

To calculate the thickness of the nerve fiber layer 102, it is necessary to detect, as detection targets, the inner limiting membrane 103 and the boundary (nerve fiber layer boundary 104) between the nerve fiber layer 102 and a layer under it, and recognize their position information.

To calculate the thickness of entire retina, it is necessary to detect, as detection targets, the inner limiting membrane 103 and the outer boundary of the retinal pigment epithelium layer 101 (retinal pigment epithelium layer boundary 105) and recognize their position information, as shown in 1b of FIG. 1A.

That is, when diagnosing the presence/absence of a disease such as glaucoma, its degree of progress, and the like, it is effective to detect the inner limiting membrane 103, nerve fiber layer boundary 104, and retinal pigment epithelium layer boundary 105 as detection targets, and calculate the nerve fiber layer thickness and the thickness of entire retina as diagnosis information data.

On the other hand, 1c in FIG. 1A shows the tomogram of a macular portion of retina of a patient suffering from age-rated macular degeneration. In a case of age-rated macular degeneration, neovascularity or drusen are generated under the retinal pigment epithelium layer 101. For this reason, the retinal pigment epithelium layer 101 is lifted, and its boundary deforms unevenly (that is, the retinal pigment epithelium layer 101 is distorted). Hence, the presence/absence of age-rated macular degeneration can be determined by determining the presence/absence of distortion of the retinal pigment epithelium layer 101 as an eye feature. Upon determining that age-rated macular degeneration exists, its degree of progress can quantitatively be diagnosed by calculating the degree of deformation of the retinal pigment epithelium layer 101 or the thickness of entire retina.

Note that when calculating the degree of deformation of the retinal pigment epithelium layer 101, first, the boundary of the retinal pigment epithelium layer 101 (retinal pigment epithelium layer boundary 105) (solid line) is detected as a detection target, and its position information is recognized, as shown in 1d of FIG. 1A. Then, the estimated position (broken line: to be referred to as a normal structure 106 hereinafter) of the boundary of the retinal pigment epithelium layer 101, which is assumed to exist in a normal state (in an assumed normal state) is detected as a detection target, and its position information is recognized. The areas of portions (hatched portions in 1d of FIG. 1A) formed by the boundary of the retinal pigment epithelium layer 101 (retinal pigment epithelium layer boundary 105) and its normal structure 106, the sum (volume) of them, and the like are calculated, thereby calculating the degree of deformation of the retinal pigment epithelium layer 101. The thickness of entire retina can be calculated by detecting the inner limiting membrane 103 and the normal structure 106 of the retinal pigment epithelium layer 101 as detection targets and recognizing their position information, as shown in 1d of FIG. 1A. Note that the area (volume) of the hatched portions in 1d of FIG. 1A will be referred to as the area (volume) of a region between the actual measured position and the estimated position of retinal pigment epithelium layer boundary hereinafter.

In this way, the eye state is determined based on the presence/absence of distortion of the retinal pigment epithelium layer 101 as an eye feature. Upon determining that age-rated macular degeneration exists, the inner limiting membrane 103, the retinal pigment epithelium layer boundary 105, and its normal structure 106 are detected as detection targets. Then, the thickness of entire retina and the degree of deformation of the retinal pigment epithelium layer 101 (the area (volume) of a region between the actual measured position and the estimated position of retinal pigment epithelium layer boundary) are effectively calculated as diagnosis information data.

On the other hand, 1e in FIG. 1A shows a tomogram of a macular portion of a patient suffering from macular edema. In a case of macular edema, the retina retains water and gets swollen. Especially, when a liquid is retained outside the cells in the retina, a clumped low-luminance region called a cyst 107 is generated, resulting in an increase in the thickness of entire retina. Hence, the presence/absence of macular edema can be determined by determining the presence/absence of the cyst 107 as an eye feature. Upon determining that macular edema exists, its degree of progress can quantitatively be diagnosed by calculating the thickness (T2 in 1e of FIG. 1A) of entire retina.

Note that as described above, when calculating the thickness T2 of entire retina, the boundary of the retinal pigment epithelium layer 101 (retinal pigment epithelium layer boundary 105) and the inner limiting membrane 103 are detected as detection targets, and their position information are recognized.

When the eye state is thus determined as macular edema based on the presence/absence of the cyst 107 as an eye feature, it is effective to detect the inner limiting membrane 103 and the retinal pigment epithelium layer boundary 105 as detection targets, and calculate the thickness of entire retina as diagnosis information data.

Note that in age-rated macular degeneration or macular edema, clumped high-luminance regions called white spots may be formed by lipid in blood accumulated in the retina (indicated by reference numeral 108 in the tomographic retina image of the patient suffering from macular edema in 1e of FIG. 1A). In the following description, when determining the eye state as age-rated macular degeneration or macular edema, the presence/absence of the white spots 108 as an eye feature is also determined.

Note that when the white spots 108 are extracted as an eye feature, measurement light is blocked, and the signal attenuates in a region deeper than the white spots 108, as shown in 1e of FIG. 1A. For this reason, upon determining age-rated macular degeneration or macular edema, the detection parameters are preferably changed in accordance with the presence/absence of white spots when detecting the retinal pigment epithelium layer boundary 105 as a detection target.

As described above, when diagnosing the presence/absence of glaucoma, age-rated macular degeneration, or macular edema and the degree of progress of each disease, the eye state is determined based on an eye feature (the presence/absence of distortion of retinal pigment epithelium layer, the presence/absence of a cyst, and the presence/absence of a white spot). It is effective to change, in accordance with the determined eye state, diagnosis information data to be acquired, detection targets to be detected, detection parameters to be set for detecting the detection targets, and the like.

In FIG. 1B is a table that provides a summary of the relationship between eye states, eye features, detection targets, and diagnosis information data. An image processing apparatus for executing image analysis processing based on the table shown in FIG. 1B will be described below in detail.

Note that in this embodiment, a case will be described in which the retinal pigment epithelium layer boundary 105 is detected as a detection target. However, the detection target is not always limited to the outer boundary of the retinal pigment epithelium layer 101 (retinal pigment epithelium layer boundary 105). For example, another layer boundary (outer limiting membrane (not shown), visual cell inner/outer segment boundary (not shown), inner boundary of the retinal pigment epithelium layer 101 (not shown), or the like) may be detected.

In addition, in this embodiment, a case will be described in which the distance between the inner limiting membrane 103 and the nerve fiber layer boundary 104 is detected as the nerve fiber layer thickness. However, the present invention is not limited to this. Instead, an outer boundary 104a of inner plexiform layer (1b in FIG. 1A) may be detected so as to calculate the distance between the inner limiting membrane 103 and the outer boundary 104a of inner plexiform layer.

<2. Configuration of Diagnostic Imaging System>

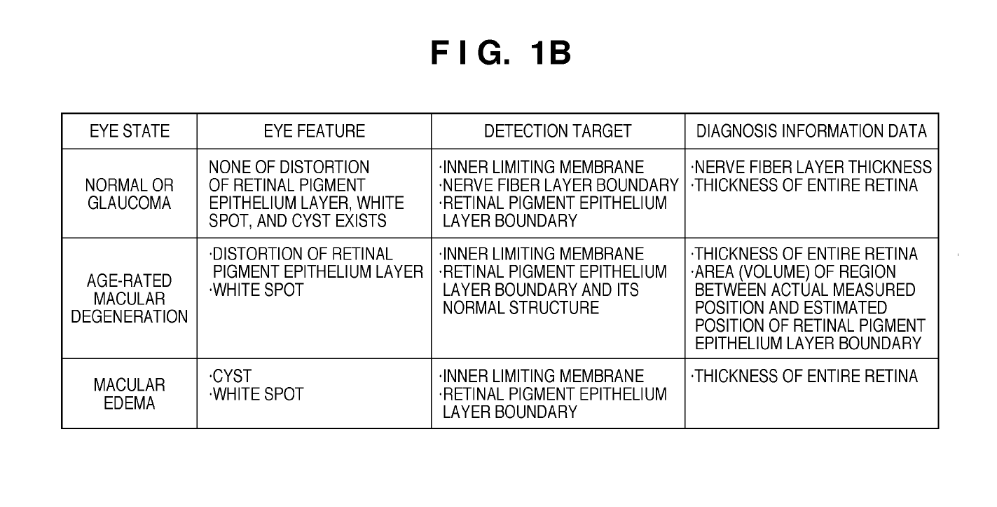

A diagnostic imaging system 200 including the image processing apparatus according to the embodiment will be described next. FIG. 2 is a block diagram showing the system configuration of the diagnostic imaging system 200 including an image processing apparatus 201 according to the embodiment.

As shown in FIG. 2, the image processing apparatus 201 is connected to a tomographic imaging apparatus 203 and a data server 202 via a local area network (LAN) 204 by Ethernet or the like. Note that the image processing apparatus may be connected to these apparatuses via an external network such as the Internet.

The tomographic imaging apparatus 203 is an apparatus for obtaining a tomogram of an eye. The apparatus includes, for example, a time domain or Fourier domain OCT. The tomographic imaging apparatus 203 obtains a three-dimensional tomogram of an eye of interest (not shown) in accordance with the operation of an operator (not shown). The obtained tomogram is transmitted to the image processing apparatus 201 or data server 202.

The data server 202 is a server for storing the tomograms of the eye of interest, its diagnosis information data, and the like. The data server 202 stores the tomograms of the eye of interest output from the tomographic imaging apparatus 203, diagnosis information data output from the image processing apparatus 201, and the like. The data server 202 also transmits the past tomograms of the eye of interest to the image processing apparatus 201 in response to a request from it.

<3. Hardware Configuration of Image Processing Apparatus>

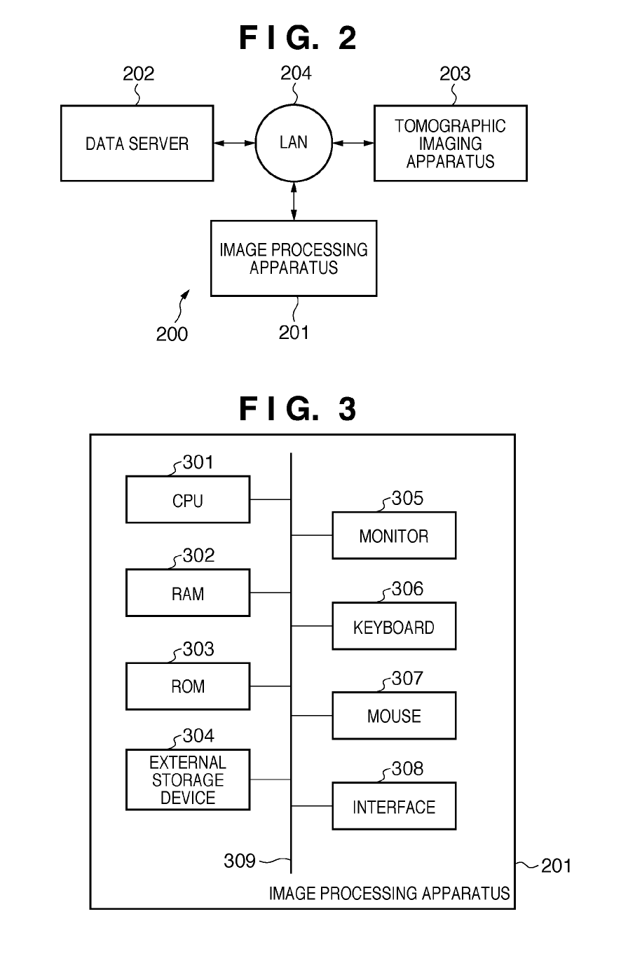

The hardware configuration of the image processing apparatus 201 according to the embodiment will be described next. FIG. 3 is a block diagram showing the hardware configuration of the image processing apparatus 201. Referring to FIG. 3, reference numeral 301 denotes a CPU; 302, a RAM; 303, a ROM; 304, an external storage device; 305, a monitor; 306, a keyboard; 307, a mouse; 308, an interface to be used to communicate with an external device (data server 202 or tomographic imaging apparatus 203); and 309, a bus.

In the image processing apparatus 201, control programs that implement the image analysis function to be described below in detail and data to be used by the control programs are stored in the external storage device 304. Note that the control programs and data are read out to the RAM 302 via the bus 309 as needed under the control of the CPU 301 and executed by the CPU 301.

<4. Functional Arrangement of Image Processing Apparatus>

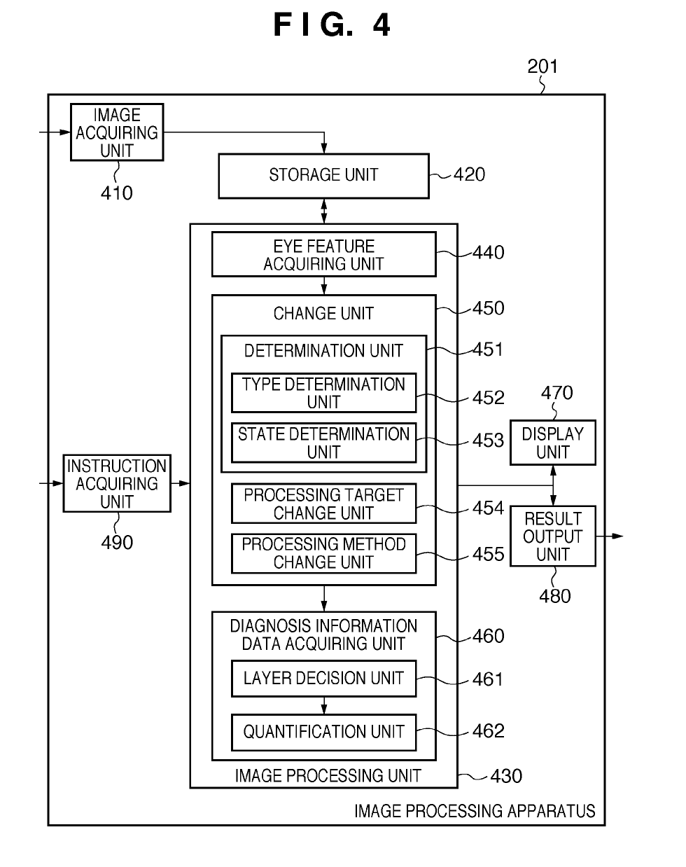

The functional arrangement of the image analysis function of the image processing apparatus 201 according to the embodiment will be described next with reference to FIG. 4. FIG. 4 is a block diagram showing the functional arrangement of the image analysis function of the image processing apparatus 201. As shown in FIG. 4, the image processing apparatus 201 includes, as the image analysis function, an image acquiring unit 410, storage unit 420, image processing unit 430, display unit 470, result output unit 480, and instruction acquiring unit 490.

Additionally, the image processing unit 430 includes an eye feature acquiring unit 440, change unit 450, and diagnosis information data acquiring unit 460. Furthermore, the change unit 450 includes a determination unit 451, processing target change unit 454, and processing method change unit 455. The determination unit 451 includes a type determination unit 452 and a state determination unit 453. On the other hand, the diagnosis information data acquiring unit 460 includes a layer decision unit 461 and a quantification unit 462. The outline of the functions of these units will be explained below.

(1) Functions of Image Acquiring Unit 410 and Storage Unit 420

The image acquiring unit 410 receives a tomogram that is an image analysis target from the tomographic imaging apparatus 203 or the data server 202 via the LAN 204, and stores it in the storage unit 420.

The storage unit 420 stores the tomogram acquired by the image acquiring unit 410. The storage unit 420 also stores eye features and detection targets to be used to determine an eye state obtained by causing the eye feature acquiring unit 440 to process the stored tomogram.

(2) Functions of Eye Feature Acquiring Unit 440

The eye feature acquiring unit 440 in the image processing unit 430 reads out the tomogram stored in the storage unit 420, and extracts the cyst 107 and white spot 108, which are eye features to be used to determine the eye state. The eye feature acquiring unit 440 also extracts the retinal pigment epithelium layer boundary 105 which is an eye feature to be used to determine the eye state and also a detection target to be used to calculate diagnosis information data. The eye feature acquiring unit 440 also extracts the inner limiting membrane 103 that is a detection target to be detected independently of the eye state.

Note that the cyst 107 or white spot 108 is extracted by an image processing method or pattern recognition method using a discriminator or the like. The eye feature acquiring unit 440 of this embodiment uses the method by a discriminator.

Note that the method of extracting the cyst 107 or white spot 108 using a discriminator is performed in accordance with the following processes (i) to (iv).

(i) feature amount calculation in a tomogram for learning
(ii) feature space creation
(iii) feature amount calculation in a tomogram of image analysis target
(iv) determination (mapping of feature amount vectors on the feature space)

More specifically, luminance information in each of the local regions of the cyst 107 and white spot 108 is acquired from the tomogram for learning to be used to extract the cyst 107 and white spot 108, and a feature amount is calculated based on the luminance information. Note that when calculating the feature amount, luminance information is acquired from a local region that is defined as a region including a pixel and its peripheral region. The feature amount calculated based on the acquired luminance information contains the statistic of luminance information in overall local regions and the statistic of luminance information of edge components of the local regions. The statistic includes the average value, maximum value, minimum value, variance, median, mode, or the like of pixel values. The edge components of the local regions include a sobel component, gabor component, and the like.

A feature space is created using the feature amount thus calculated based on the tomogram for learning. After that, a feature amount is calculated for the tomogram of image analysis target in accordance with the same procedure and mapped on the created feature space.

With this processing, the eye features extracted from the tomogram of image analysis target are classified as the white spot 108, cyst 107, retinal pigment epithelium layer 101, and the like. Note that in this classification, the eye feature acquiring unit 440 uses a feature space created using a self-organizing map.

Note that although a method of classifying eye features using a self-organizing map has been described here, the present invention is not limited to this. An arbitrary known discriminator such as SVM (Support Vector Machine) or AdaBoost is also usable.

The method of classifying eye features such as the white spot 108 and cyst 107 is not limited to the above-described method. The eye features may be classified by image processing. For example, following classification can be executed by combining luminance information and the output value from a filter such as a point convergence index filter for emphasizing a clumped structure. More specifically, classification can be executed by determining a region where the point convergence index filter output is equal to or more than a threshold Tc1, and the luminance value on the tomogram is equal to or more than a threshold Tg1 as a white spot, and a region where the point convergence index filter output is equal to or more than a threshold Tc2, and the luminance value on the tomogram is less than a threshold Tg2 as a cyst.

On the other hand, the eye feature acquiring unit 440 extracts the retinal pigment epithelium layer boundary 105 and the inner limiting membrane 103 in accordance with the following procedure. Note that in this extraction, the three-dimensional tomogram of image analysis target is regarded as a set of two-dimensional tomograms (B scan images), and the following processing is executed for each two-dimensional tomogram.

First, smoothing processing is performed for a two-dimensional tomogram of interest to remove noise components. Next, edge components are extracted from the two-dimensional tomogram. Several line segments are extracted as layer boundary candidates based on the connectivity. Out of the plurality of layer boundary candidates, the uppermost line segment is selected as the inner limiting membrane 103. In addition, the lowermost line segment is selected as the retinal pigment epithelium layer boundary 105.

However, the extraction procedure of the retinal pigment epithelium layer boundary 105 is merely an example, and is not limited to this. For example, a deformable model such as Snakes or level set method may be applied while defining a thus selected line segment as the initial value, thereby determining a finally selected line segment as the retinal pigment epithelium layer boundary 105 or the inner limiting membrane 103. Alternatively, a graph cuts method may be used for extraction. Note that the extraction method using a deformable model or graph cuts may be executed three-dimensionally for a three-dimensional tomogram or two-dimensionally for each two-dimensional tomogram. The retinal pigment epithelium layer boundary 105 or the inner limiting membrane 103 can be extracted by any other method capable of extracting a layer boundary from a tomogram of an eye.

(3) Functions of Determination Unit 451 in Change Unit 450

The change unit 450 determines the eye state based on the eye features extracted by the eye feature acquiring unit 440, and also instructs, based on the determined eye state, to change the image analysis algorithm to be executed by the diagnosis information data acquiring unit 460.

The determination unit 451 included in the change unit 450 determines the eye state based on the eye features extracted by the eye feature acquiring unit 440. More specifically, the type determination unit 452 determines the presence/absence of the cyst 107 or white spot 108 based on the eye feature classification result from the eye feature acquiring unit 440. In addition, the state determination unit 453 determines the presence/absence of distortion of the retinal pigment epithelium layer boundary 105 classified by the eye feature acquiring unit 440, and also determines the eye state based on that determination result and the determination result of the presence/absence of the cyst 107 and white spot 108.

(4) Functions of Processing Target Change Unit 454 and Processing Method Change Unit 455 in Change Unit 450

On the other hand, the processing target change unit 454 included in the change unit 450 changes the detection target in accordance with the eye state determined by the state determination unit 453. The processing target change unit 454 also notifies the layer decision unit 461 of information about the changed detection target.

When the state determination unit 453 determines that the white spot 108 has been extracted, the processing method change unit 455 instructs the layer decision unit 461 to change the detection parameters of the retinal pigment epithelium layer boundary 105 in a region deeper than the region where the white spot 108 exists. When it is determined that the retinal pigment epithelium layer boundary 105 has distortion, the processing method change unit 455 instructs the layer decision unit 461 to change the detection parameters of the distorted portion of the retinal pigment epithelium layer boundary.

That is, if it is determined that the eye state is age-rated macular degeneration or macular edema, the processing method change unit 455 instructs the layer decision unit 461 to change the detection parameters so as to more accurately detect (redetect) the retinal pigment epithelium layer boundary 105.

(5) Functions of Diagnosis Information Data Acquiring Unit 460

The diagnosis information data acquiring unit 460 calculates diagnosis information data using the detection targets extracted by the eye feature acquiring unit 440 and, upon receiving an instruction from the processing method change unit 455, the detection target extracted based on the instruction as well.

The layer decision unit 461 acquires the detection targets detected by the eye feature acquiring unit 440 and stored in the storage unit 420. Note that upon receiving a change instruction for a detection target from the processing method change unit 455, the layer decision unit 461 detects the designated detection target, and then acquires the detection targets. Upon receiving a detection parameter change instruction from the processing method change unit 455, the layer decision unit 461 detects (redetects) the detection target again using the changed detection parameters, and then acquires the detection targets. The layer decision unit 461 also calculates the normal structure 106 of the retinal pigment epithelium layer boundary.

The quantification unit 462 calculates diagnosis information parameters based on the detection targets acquired by the layer decision unit 461.

More specifically, the quantification unit 462 quantifies the thickness of the nerve fiber layer 102 and the thickness of entire retinal layer based on the nerve fiber layer boundary 104. Note that in this quantification, first, the difference in z-coordinate between the nerve fiber layer boundary 104 and the inner limiting membrane 103 is obtained at each coordinate point on the x-y plane, thereby calculating the thickness of the nerve fiber layer 102 (T1 in 1a of FIG. 1A). Similarly, the difference in z-coordinate between the inner limiting membrane 103 and the retinal pigment epithelium layer boundary 105 is obtained, thereby calculating the thickness of entire retinal layer (T2 in 1a of FIG. 1A). In addition, the thicknesses at the coordinate points in the x-axis direction are added for each y-coordinate so as to calculate the area of each of the layers (nerve fiber layer 102 and the entire retinal layer) along each section. Then, the obtained areas are added in the y-axis direction to calculate the volume of each layer. Furthermore, the area or volume of the portion formed between the retinal pigment epithelium layer boundary 105 and the normal structure 106 of the retinal pigment epithelium layer boundary (the area or volume of a region between the actual measured position and the estimated position of retinal pigment epithelium layer boundary) is calculated.

(6) Functions of Display Unit 470, Result Output Unit 480, and Instruction Acquiring Unit 490

The display unit 470 displays the detected nerve fiber layer boundary 104 by superimposing it on the tomogram. The display unit 470 also displays quantified diagnosis information data. Out of the diagnosis information data, information about the layer thickness may be displayed as a layer thickness distribution map of the entire three-dimensional tomogram (x-y plane), or as the area of each layer on the section of interest in synchronism with the above-described detection result display. Alternatively, the volume of each layer or the volume of a region designated on the x-y plane by the operator may be calculated and displayed.

The result output unit 480 transmits the imaging date/time, the image analysis processing result (diagnosis information data) obtained by the image processing unit 430, and the like to the data server 202 in association with each other.

The instruction acquiring unit 490 receives, from outside, an instruction to end or not to end the image analysis processing of the tomogram by the image processing apparatus 201. Note that the instruction is input by the operator via the keyboard 306, mouse 307, or the like.

Procedure of Image Analysis Processing of Image Processing Apparatus>

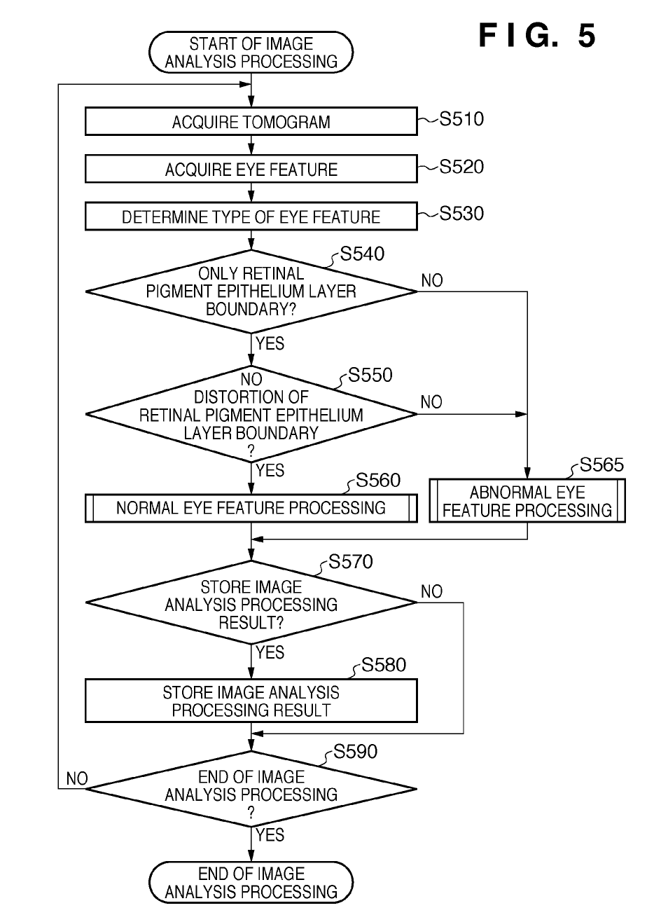

The procedure of image analysis processing of the image processing apparatus 201 will be described next. FIG. 5 is a flowchart illustrating the procedure of image analysis processing of the image processing apparatus 201.

In step S510, the image acquiring unit 410 transmits a tomogram acquisition request to the tomographic imaging apparatus 203. The tomographic imaging apparatus 203 transmits a corresponding tomogram in response to the acquisition request. The image acquiring unit 410 receives the transmitted tomogram via the LAN 204. Note that the tomogram received by the image acquiring unit 410 is stored in the storage unit 420.

In step S520, the eye feature acquiring unit 440 reads out the tomogram stored in the storage unit 420, and extracts the inner limiting membrane 103, retinal pigment epithelium layer boundary 105, white spot 108, and cyst 107 from the tomogram. The extracted eye features are stored in the storage unit 420.

In step S530, the type determination unit 452 classifies the eye features extracted in step S520 as the white spot 108, cyst 107, retinal pigment epithelium layer boundary 105, and the like.

In step S540, the state determination unit 453 determines the eye state based on the result of eye feature classification performed by the type determination unit 452 in step S530. More specifically, upon determining that the eye features include only the retinal pigment epithelium layer boundary 105 (neither the cyst 107 nor the white spot 108 exists on the tomogram), the state determination unit 453 determines it as a first state, and advances to step S550. On the other hand, upon determining that the eye features include the white spot 108 or cyst 107, the state determination unit 453 advances to step S565.

In step S550, the state determination unit 453 determines the presence/absence of distortion of the retinal pigment epithelium layer boundary 105 classified by the type determination unit 452 in step S530.

Upon determining in step S550 that the retinal pigment epithelium layer boundary 105 has no distortion, the process advances to step S560. Upon determining in step S550 that the retinal pigment epithelium layer boundary 105 has distortion, the process advances to step S565.

In step S560, the diagnosis information data acquiring unit 460 executes an image analysis algorithm (normal eye feature processing) for a case in which neither the cyst 107 nor the white spot 108 exists, and the retinal pigment epithelium layer boundary 105 has no distortion (when the eye features are normal). In other words, the normal eye feature processing is processing of calculating diagnosis information data effective for quantitatively diagnosing the presence/absence of glaucoma, the degree of progress of glaucoma, and the like. Note that the normal eye feature processing will be described later in detail.

On the other hand, in step S565, the image processing unit 430 executes an image analysis algorithm (abnormal eye feature processing) for a case in which the cyst 107, the white spot 108, or distortion of the retinal pigment epithelium layer boundary 105 exists (that is, when the eye features are abnormal). In other words, the abnormal eye feature processing is processing of calculating diagnosis information data effective for quantitatively diagnosing the presence/absence of age-rated macular degeneration or macular edema, its degree of progress, and the like. Note that the abnormal eye feature processing will be described later in detail.

In step S570, the instruction acquiring unit 490 acquires, from outside, an instruction to store or not to store the current image analysis processing result for the eye of interest in the data server 202. This instruction is input by the operator via, for example, the keyboard 306 or mouse 307. Upon acquiring an instruction to store, the process advances to step S580. If no instruction to store has been acquired, the process advances to step S590.

In step S580, the result output unit 480 transmits the imaging date/time, information to identify the eye of interest, the tomogram, and the image analysis processing result obtained by the image processing unit 430 to the data server 202 in association with each other.

In step S590, the instruction acquiring unit 490 determines whether an instruction to end the image analysis processing of the tomogram by the image processing apparatus 201 has been acquired from outside. Upon determining that an instruction to end the image analysis processing has been acquired, the image analysis processing ends. On the other hand, upon determining that no instruction to end the image analysis processing has been acquired, the process returns to step S510 to perform processing of the next eye of interest (or reprocessing of the same eye of interest).

<6. Procedure of Normal Eye Feature Processing>

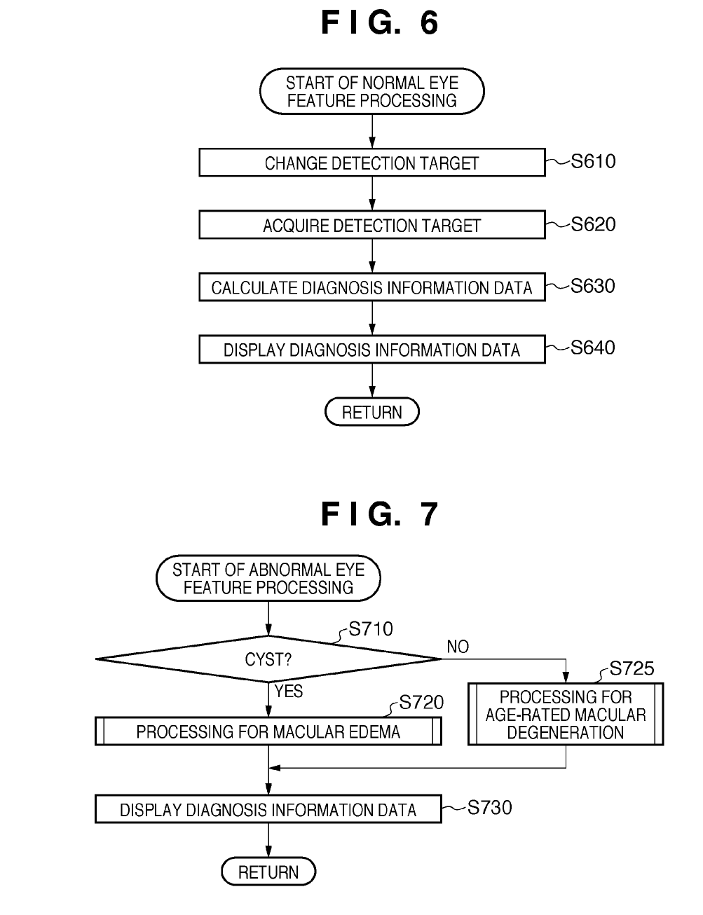

The normal eye feature processing (step S560) will be described next in detail with reference to FIG. 6.

In step S610, the processing target change unit 454 instructs to change the detection target. More specifically, the processing target change unit 454 instructs to newly detect the nerve fiber layer boundary 104 as a detection target. Note that the instruction concerning the detection target is not limited to this, and an instruction to newly detect, for example, the outer boundary 104a of inner plexiform layer may be issued.

In step S620, the layer decision unit 461 detects the detection target designated in step S610, that is, the nerve fiber layer boundary 104 from the tomogram, and also acquires already detected detection targets (inner limiting membrane 103 and retinal pigment epithelium layer boundary 105) from the storage unit 420. Note that the nerve fiber layer boundary 104 is detected by, for example, scanning the z-coordinate values of the inner limiting membrane 103 in the positive z-axis direction to extract points whose luminance value or edge is equal to or more than a threshold and connecting the extracted points.

In step S630, the quantification unit 462 quantifies the thickness of the nerve fiber layer 102 and the thickness of entire retinal layer based on the detection targets acquired in step S620 (calculates diagnosis information data). More specifically, first, the difference in z-coordinate between the nerve fiber layer boundary 104 and the inner limiting membrane 103 is obtained at each coordinate point on the x-y plane, thereby calculating the thickness of the nerve fiber layer 102 (T1 in 1a of FIG. 1A). Similarly, the difference in z-coordinate between the inner limiting membrane 103 and the retinal pigment epithelium layer boundary 105 is obtained, thereby calculating the thickness of entire retinal layer (T2 in 1a of FIG. 1A). In addition, the thicknesses at the coordinate points in the x-axis direction are added for each y-coordinate so as to calculate the area of each of the layers (nerve fiber layer 102 and the entire retinal layer) along each section. Furthermore, the volume of each layer is calculated by adding the obtained areas in the y-axis direction.

In step S640, the display unit 470 displays the nerve fiber layer boundary 104 acquired in step S620 by superimposing it on the tomogram. The display unit 470 also displays the diagnosis information data (the nerve fiber layer thickness and the thickness of entire retinal layer) obtained by quantification in step S630. This display may be presented as a layer thickness distribution map of the entire three-dimensional tomogram (x-y plane), or as the area of each layer on the section of interest in synchronism with display of the above-described detection target acquisition result. Alternatively, the volume of each layer or the volume of each layer in a region designated on the x-y plane by the operator may be calculated and displayed.

<7. Procedure of Abnormal Eye Feature Processing>

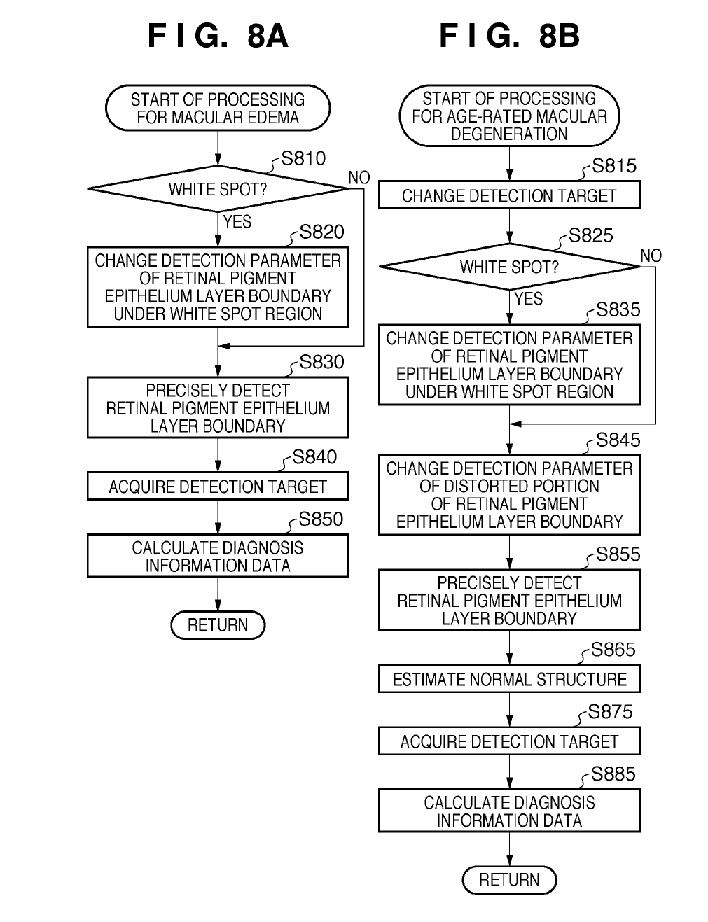

The abnormal eye feature processing (step S565) will be described next in detail. FIG. 7 is a flowchart illustrating the procedure of abnormal eye feature processing.

In step S710, the state determination unit 453 determines the eye state based on the result of eye feature classification performed by the type determination unit 452 in step S530. More specifically, if it is determined in step S530 that the eye features include the cyst 107, the state determination unit 453 determines that the eye state is macular edema (third state), and advances to step S720. On the other hand, if it is determined in step S530 that the eye features include no cyst 107, the state determination unit 453 determines that the eye state is age-rated macular degeneration (second state), and advances to step S725.

In step S720, the layer decision unit 461 and the quantification unit 462 perform processing (processing for macular edema) of calculating diagnosis information data effective for diagnosing the degree of progress of macular edema or the like. Note that the processing for macular edema will be described later in detail.

On the other hand, in step S725, the layer decision unit 461 and the quantification unit 462 perform processing (processing for age-rated macular degeneration) of calculating diagnosis information data effective for diagnosing the degree of progress of age-rated macular degeneration or the like. Note that the processing for age-rated macular degeneration will be described later in detail.

In step S730, the display unit 470 displays the detection targets acquired in step S720 or the diagnosis information data calculated in step S725. Note that this processing is the same as that in step S640, and a detailed description thereof will not be repeated here.

<8. Details of Processing for Macular Edema>

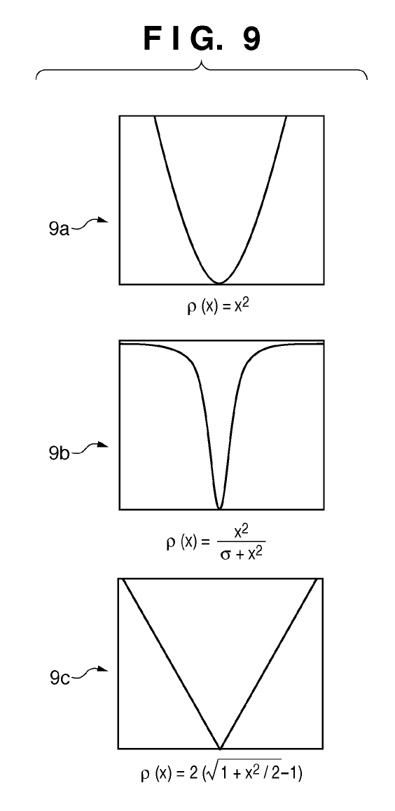

The processing for macular edema (step S720) will be described next in detail. FIG. 8A is a flowchart illustrating the procedure of processing for macular edema.

In step S810, the processing method change unit 455 branches the process based on the result of eye feature classification performed by the type determination unit 452 in step S530. If the white spot 108 is included as an eye feature, as described above with reference to 1e in FIG. 1A, the white spot 108 blocks measurement light. Consequently, the luminance value attenuates in a region having coordinate values larger than those of the white spot 108 in the depth direction (z-axis direction) (109 in 1e of FIG. 1A). Hence, the detection parameters for detection of the retinal pigment epithelium layer boundary 105 are changed in a region that has the same coordinate values as those of the white spot 108 in the horizontal direction (x-axis direction) of the B scan image and is deeper than the white spot 108.

More specifically, if the eye features include the white spot 108, the processing method change unit 455 instructs the layer decision unit 461 to change the detection parameters of the retinal pigment epithelium layer boundary 105 in a region deeper than the region where the white spot 108 exists. Then, the process advances to step S820. On the other hand, if the eye features include no white spot 108, the process advances to step S830.

In step S820, the layer decision unit 461 sets the detection parameters of the retinal pigment epithelium layer boundary 105 in the region deeper than the region where the white spot 108 exists in the following way. In this case, a deformable model is used as the detection method.

That is, the weight of image energy (evaluation function concerning the luminance value) is increased in accordance with the degree of attenuation of the luminance value in the region 109 where the luminance value attenuates. More specifically, a value proportional to a ratio T/F of a luminance statistic F in the region 109 where the luminance value attenuates to a luminance statistic T in the region where the luminance value does not attenuate is set as the weight of image energy.

Note that although a case in which the detection parameters are changed has been described, the processing of the layer decision unit 461 is not limited to this. For example, the detection method itself may be changed so as to execute the deformable model after image correction in the region 109 where the luminance value attenuates.

In step S830, the quantification unit 462 detects the retinal pigment epithelium layer boundary 105 again based on the detection parameters set in step S820.

In step S840, the already detected detection target (inner limiting membrane 103) is acquired from the storage unit 420.

In step S850, the quantification unit 462 calculates the thickness of entire retina based on the retinal pigment epithelium layer boundary 105 detected in step S830 and the inner limiting membrane 103 acquired in step S840. Note that the process of step S850 is the same as that of step S630, and a detailed description thereof will not be repeated here.

<9. Details of Processing for Age-Rated Macular Degeneration>

The processing for age-rated macular degeneration (step S725) will be described next in detail. FIG. 8B is a flowchart illustrating the procedure of processing for age-rated macular degeneration.

In step S815, the processing target change unit 454 instructs to change the detection target. More specifically, the processing target change unit 454 instructs to newly detect the normal structure 106 of the retinal pigment epithelium layer boundary as a detection target.

In step S825, the processing method change unit 455 branches the process. More specifically, if the white spot 108 is included as an eye feature, the processing method change unit 455 instructs the layer decision unit 461 to change the detection parameters of the retinal pigment epithelium layer boundary 105 in a region deeper than the region where the white spot 108 exists.

On the other hand, if neither the white spot 108 nor distortion of the retinal pigment epithelium layer boundary 105 is included as an eye feature, the process advances to step S845.

In step S835, the layer decision unit 461 changes the detection parameters of the retinal pigment epithelium layer boundary 105 in the region deeper than the region where the white spot 108 exists. The processing of changing the detection parameters in the region deeper than the region where the white spot 108 exists is the same as the process of step S820, and a detailed description thereof will not be repeated here.

In step S845, the processing method change unit 455 instructs the layer decision unit 461 to change the detection parameters of the distorted portion of the retinal pigment epithelium layer boundary. This is because when distortion of the retinal pigment epithelium layer boundary 105 is included as an eye feature, the degree of distortion serves as an indicator to be used to diagnose the degree of progress of age-rated macular degeneration, and the retinal pigment epithelium layer boundary 105 needs to be obtained more accurately. To do this, the processing target change unit 454 first designates a range of the retinal pigment epithelium layer boundary 105 where distortion exists. Then, the processing target change unit 454 instructs the layer decision unit 461 to change the detection parameters of the retinal pigment epithelium layer boundary 105 in the designated range. The layer decision unit 461 changes the detection parameters of the distorted portion of the retinal pigment epithelium layer boundary.

Note that the processing of changing the detection parameters in a region of the retinal pigment epithelium layer boundary 105 determined to have distortion is executed in the following way. A case will be explained below in which the Snakes method is used to detect the region of the retinal pigment epithelium layer boundary 105 including distortion.

More specifically, the weight of shape energy of the layer boundary model corresponding to the retinal pigment epithelium layer boundary 105 is set to be smaller than image energy. This allows to more accurately acquire distortion of the retinal pigment epithelium layer boundary 105. That is, the indicator representing distortion of the retinal pigment epithelium layer boundary 105 is calculated, and a value proportional to the indicator is set as the weight of shape energy.

Note that although in this embodiment, the weights of evaluation functions (shape energy and image energy) to be used to deform the layer boundary model are set to be variable at each control point of the layer, the present invention is not limited to this. For example, the weights of shape energy at all control points of the retinal pigment epithelium layer boundary 105 may be set to be uniformly smaller than image energy.

Referring back to FIG. 8B, in step S855, the layer decision unit 461 detects the retinal pigment epithelium layer boundary 105 again based on the detection parameters set in steps S835 and S845.

In step S865, the layer decision unit 461 estimates the normal structure 106 based on the retinal pigment epithelium layer boundary 105 detected in step S855. Note that when estimating the normal structure 106, the three-dimensional tomogram of image analysis target is regarded as a set of two-dimensional tomograms (B scan images), and normal structure estimation is done for each two-dimensional tomogram.

More specifically, the normal structure 106 is estimated by applying a quadratic function to a coordinate point group representing the retinal pigment epithelium layer boundary 105 detected in each two-dimensional tomogram.

Let εi be the difference between a z-coordinate zi of the ith point of layer boundary data of the retinal pigment epithelium layer boundary 105 and a z-coordinate z′i of the ith point of the normal structure 106. An evaluation expression to be used to obtain an approximation function is given by, for example,

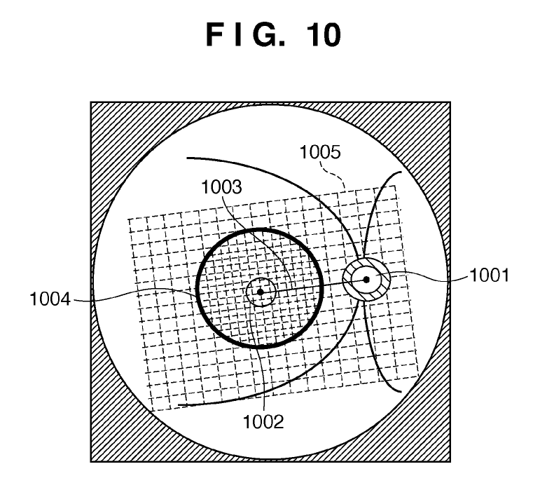

M=minΣρ(εi)

where Σ is the sum for i, and ρ( ) is a weight function. In FIGS. 9, 9a to 9c show three kinds of weight functions. Referring to 9a to 9c in FIG. 9, the abscissa represents x, and the ordinate represents ρ(x). Note that the weight functions are not limited to those shown in 9a to 9c of FIG. 9, and any other function may be set. The function is set to minimize the evaluation value M of the above expression.

Note that although in the above-described case, the input three-dimensional tomogram is regarded as a set of two-dimensional tomograms (B scan images), and the normal structure 106 is estimated in each two-dimensional tomogram, the method of estimating the normal structure 106 is not limited to this. For example, the processing may directly be executed for the three-dimensional tomogram. In this case, using the same weight function selection criterion as described above, an ellipse is applied to the three-dimensional coordinate point group of the layer boundary detected in step S530.

In the above-described case, a quadratic function is used as the shape to approximate when estimating the normal structure 106. However, the shape to approximate the normal structure 106 is not limited to the quadratic function, and the estimation can be done using an arbitrary function.

Referring back to FIG. 8B again, in step S875, the already detected detection target (inner limiting membrane 103) is acquired from the storage unit 420.

In step S885, the quantification unit 462 quantifies the thickness of entire retinal layer based on the retinal pigment epithelium layer boundary 105 detected in step S855 and the inner limiting membrane acquired in step S875. In addition, the quantification unit 462 quantifies distortion of the retinal pigment epithelium layer 101 based on the difference between the retinal pigment epithelium layer boundary 105 detected in step S855 and the normal structure 106 estimated in step S865. More specifically, the quantification is done by obtaining the sum of differences and the statistics (maximum value and the like) of the angles between layer boundary points.

As is apparent from the above description, the image processing apparatus according to the embodiment is configured to extract eye features to be used to determine the eye state in image analysis processing of an acquired tomogram. The apparatus is configured to determine the eye state based on the extracted eye features, and change a detection target to be detected from the tomogram or detection parameters for detection in accordance with the determined eye state.

Executing an image analysis algorithm corresponding to the eye state makes it possible to accurately calculate, independently of the eye state, diagnosis information parameters effective for diagnosing the presence/absence of diseases such as glaucoma, age-rated macular degeneration, and macular edema and the degree of progress of the diseases.

### Second Embodiment

In the above-described first embodiment, assuming that the image analysis target is a tomogram of a macular portion, eye features are extracted, and the eye state is determined based on the extracted eye features. However, the tomogram of image analysis target is not limited to the tomogram of a macular portion. It may be, for example, a wide-angle tomogram including not only a macular portion but also an optic disc portion. In the second embodiment, an image processing apparatus will be described which, when the tomogram of image analysis target is a wide-angle tomogram including a macular portion and an optic disc portion, specifies each part and executes an image analysis algorithm for each part.

Note that the overall arrangement of the diagnostic imaging system and the hardware configuration of the image processing apparatus are the same as in the first embodiment, and a description thereof will not be repeated here.

<1. About Wide-Angle Tomogram Including Macular Portion and Optic Disc Portion>

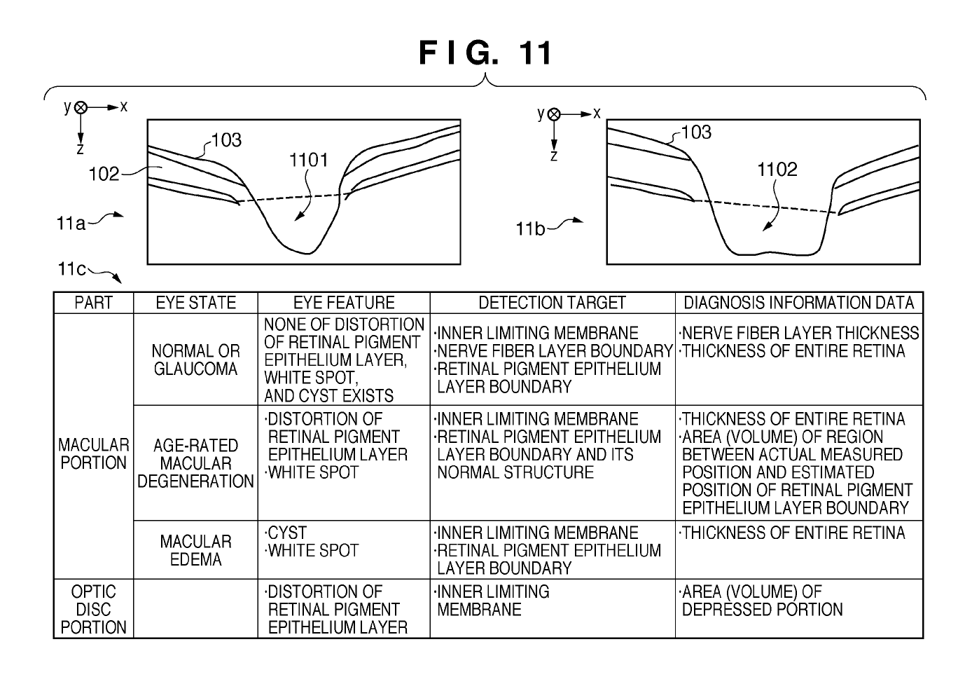

A wide-angle tomogram including a macular portion and an optic disc portion will be explained first. FIG. 10 is a view showing an imaging range on the x-y plane when obtaining a wide-angle tomogram including a macular portion and an optic disc portion.

Referring to FIG. 10, reference numeral 1001 denotes an optic disc portion; and 1002, a macular portion. As the anatomical characteristics of the optic disc portion 1001, the depth of an inner limiting membrane 103 is maximum at its center and fovea (that is, a depressed portion is formed), and blood vessels of retina exist.

On the other hand, as the anatomical characteristics of the macular portion 1002, it is present at a position apart from the optic disc portion 1001 by about twice the optic disc diameter, and the depth of the inner limiting membrane 103 is maximum at its center and fovea (that is, a depressed portion is formed). The macular portion 1002 additionally has anatomical characteristics representing that no blood vessel of retina exists, and the nerve fiber layer thickness is zero at its fovea.

To specify the optic disc portion and the macular portion from a tomogram, these anatomical characteristics are used. Note that when calculating diagnosis information data in image analysis processing of a wide-angle tomogram, a coordinate system to be described below is set on the x-y plane.

Ganglion cells are generally known to anatomically run symmetrically about a line segment 1003 that connects the optic disc portion 1001 and the macular portion 1002. In a tomogram of an eye of a normal patient, the nerve fiber layer thickness distribution is also symmetric about the line segment 1003. Hence, an orthogonal coordinate system 1005 is set by defining the line that connects the optic disc portion 1001 and the macular portion 1002 as the abscissa and an axis perpendicular to the abscissa as the ordinate, as shown in FIG. 10.

<2. Relationship between Eye States, Eye Features, Detection Targets, and Diagnosis Information Data of Respective Parts>

The relationship between eye states, eye features, detection targets, and diagnosis information data of the respective parts will be described next. Note that the relationship between eye states, eye features, detection targets, and diagnosis information data of the macular portion has already been described in the first embodiment with reference to FIGS. 1A and 1B, and a description thereof will not be repeated here. The relationship between eye states, eye features, detection targets, and diagnosis information data of the optic disc portion will be described below mainly regarding the differences from the macular portion.

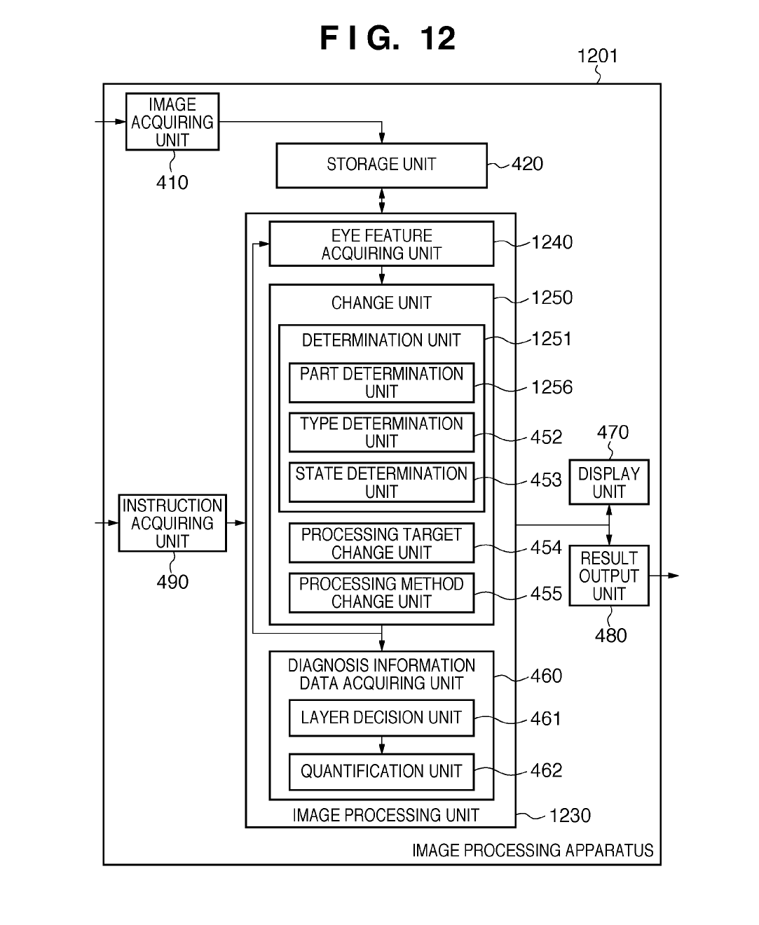

In FIGS. 11, 11a and 11b are schematic views of a tomogram of the optic disc portion of retina obtained by an OCT (an enlarged view of the inner limiting membrane 103). Referring to 11a and 11b in FIG. 11, reference numeral 1101 or 1102 denotes a depressed portion of the optic disc portion. To specify the macular portion and the optic disc portion, the image processing apparatus according to the embodiment extracts the depressed portion of each part. Hence, the image processing apparatus according to the embodiment is configured to output the quantified shape of the depressed portion as diagnosis information data of the optic disc portion. More specifically, the apparatus is configured to calculate the area or volume of the depressed portion 1101 or 1102 as diagnosis information data.

In FIG. 11, 11c is a table that provides a summary of the relationship between eye states, eye features, detection targets, and diagnosis information data of the respective parts. An image processing apparatus for executing image analysis processing based on the table shown in 11c of FIG. 11 will be described below in detail.

<3. Functional Arrangement of Image Processing Apparatus>

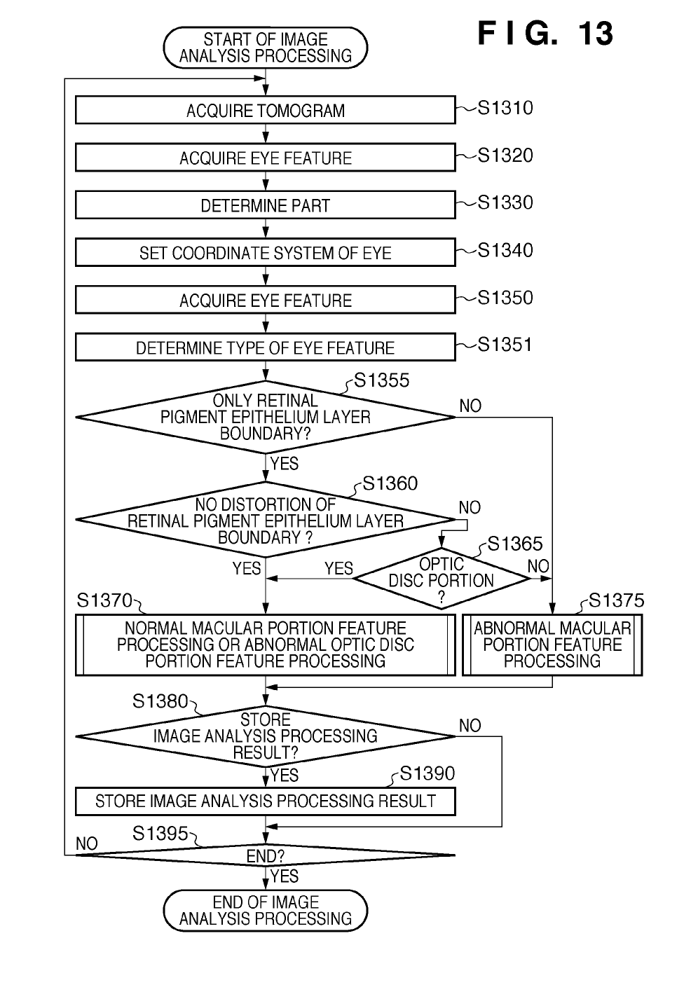

FIG. 12 is a block diagram showing the functional arrangement of the image processing apparatus according to the embodiment. This apparatus is different from the image processing apparatus 201 (FIG. 4) according to the first embodiment in that a determination unit 1251 includes a part determination unit 1256. In addition, an eye feature acquiring unit 1240 extracts eye features for part determination by the part determination unit 1256 as well as eye features for eye state determination. The functions of the eye feature acquiring unit 1240 and the part determination unit 1256 will be described below.

(1) Functions of Eye Feature Acquiring Unit 1240

The eye feature acquiring unit 1240 reads out a tomogram from a storage unit 420, like the eye feature acquiring unit 440 of the first embodiment, and extracts not only the inner limiting membrane 103 and a nerve fiber layer boundary 104 but also blood vessels of retina as eye features to be used for part determination. The blood vessels of retina are extracted by an arbitrary known enhancement filter to a plane on which the tomogram is projected in the depth direction.

(2) Functions of Part Determination Unit 1256

The part determination unit 1256 determines an anatomical part of the eye based on the eye features extracted by the eye feature acquiring unit 1240 for part determination, thereby specifying the optic disc portion and the macular portion. More specifically, the following processing is performed to determine the position of the optic disc portion.

First, a position (x- and y-coordinates) where the depth of the inner limiting membrane 103 is maximum is obtained. Since the depth exhibits the maximum value at the center and fovea in both the optic disc portion and the macular portion, the presence/absence of blood vessels of retina near the maximum value portion, that is, within the depressed portion is checked as a characteristic feature to distinguish the portions. If blood vessels of retina exist, the part is determined as the optic disc portion.

Next, the macular portion is specified. As described above, as the anatomical characteristics of the macular portion,

(i) it is present at a position apart from the optic disc portion by about twice the optic disc diameter,
(ii) no blood vessels of retina exist at the fovea (center of the macular portion),
(iii) the nerve fiber layer thickness is zero at the fovea (center of the macular portion), and
(iv) a depressed portion exists near the fovea.

((iv) does not always hold in a case of macular edema or the like).

Hence, the nerve fiber layer thickness, the presence/absence of blood vessels of retina, and the z-coordinate of the inner limiting membrane are obtained in the region part from the optic disc portion by about twice the optic disc diameter. A region where no blood vessels of retina exist, and the nerve fiber layer thickness is zero is specified as the macular portion. Note that if there are a plurality of regions that satisfy the above-described conditions, a region located on an ear side (the x-coordinate is smaller than that of the depressed portion of the optic disc for the right eye, and larger for the left eye) slightly below (inferior) the depressed portion of the optic disc is selected as the macular portion.

<4. Procedure of Image Analysis Processing of Image Processing Apparatus>

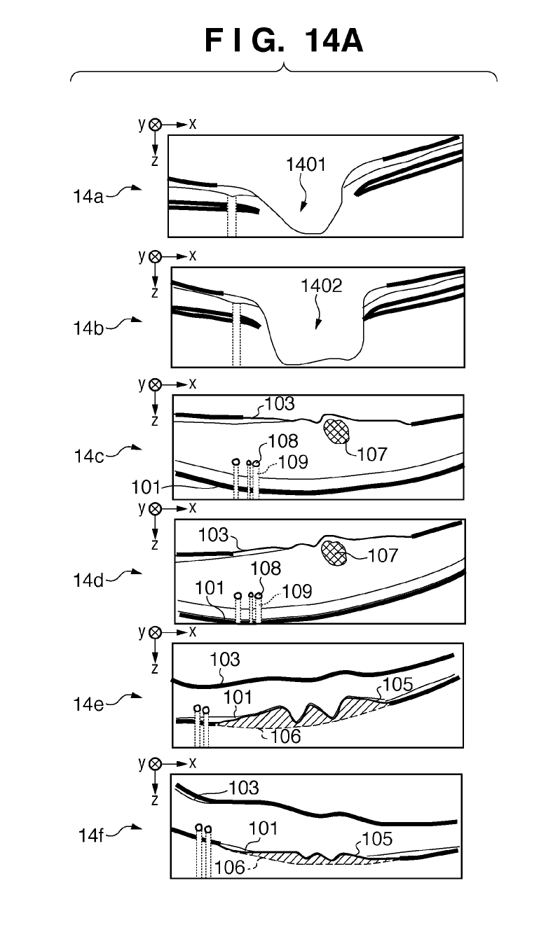

The procedure of image analysis processing of an image processing apparatus 1201 will be described next. FIG. 13 is a flowchart illustrating the procedure of image analysis processing of the image processing apparatus 1201. The processing is different from image analysis processing of the image processing apparatus 201 according to the first embodiment (FIG. 5) only in the processes of steps S1320 to S1375. The processes of steps S1320 to S1375 will be explained below.

In step S1320, the eye feature acquiring unit 1240 extracts the inner limiting membrane 103 and the nerve fiber layer boundary 104 from the tomogram as eye features for part determination. The eye feature acquiring unit 1240 also extracts blood vessels of retina from an image obtained by projecting the tomogram in the depth direction.

In step S1330, the part determination unit 1256 determines anatomical parts based on the eye features extracted in step S1320, thereby specifying the optic disc portion and the macular portion.

In step S1340, the part determination unit 1256 sets a coordinate system on the wide-angle tomogram of image analysis target based on the positions of the optic disc portion and the macular portion specified in step S1330. More specifically, as shown in FIG. 10, the orthogonal coordinate system 1005 is set by defining the line that connects the optic disc portion 1001 and the macular portion 1002 as the abscissa and an axis perpendicular to the abscissa as the ordinate.

In step S1350, based on the coordinate system set in step S1340, the eye feature acquiring unit 1240 extracts eye features to be used to determine the eye state for each part. For the optic disc portion, a retinal pigment epithelium layer boundary within a predetermined range from the center of the optic disc is extracted as an eye feature. On the other hand, for the macular portion, a retinal pigment epithelium layer boundary 105, cyst 107, and white spot 108 are extracted, as in the first embodiment. Note that the eye feature search range of the macular portion is set within a predetermined range (search range 1004 (FIG. 10)) from the fovea of the macular portion. However, the search range may be changed in accordance with the type of eye feature. For example, the white spot 108 is formed as lipid or the like leaked from the blood vessels of retina is accumulated, and does not therefore always occur in the macular portion. For this reason, the search range of white spots is set to be wider than those of other eye features.

Note that the eye feature acquiring unit 1240 need not always be configured to execute eye feature extraction within the search range 1004 based on the same processing parameter (for example, processing interval). For example, in the favorite site of age-rated macular degeneration or a part largely affecting the vision (search range 1004 or macular portion 1002 in FIG. 10), extraction may be executed by setting a narrower processing interval. This enables to execute efficient image analysis processing.

In step S1351, a type determination unit 452 classifies the eye features extracted in step S1350 as the white spot 108, cyst 107, retinal pigment epithelium layer boundary 105, and the like, thereby determining the types of eye features.

In step S1355, a state determination unit 453 determines the eye state based on the result of eye feature classification performed by the type determination unit 452 in step S1351. More specifically, upon determining that the eye features include only the retinal pigment epithelium layer boundary 105 (neither the cyst 107 nor the white spot 108 exists on the tomogram), the state determination unit 453 advances to step S1360. On the other hand, upon determining that the eye features include the white spot 108 or cyst 107, the state determination unit 453 advances to step S1375.

In step S1360, the state determination unit 453 determines the presence/absence of distortion of the retinal pigment epithelium layer boundary 105 classified by the type determination unit 452 in step S1351.

Upon determining in step S1360 that the retinal pigment epithelium layer boundary 105 has no distortion, the process advances to step S1370.

Upon determining in step S1360 that the retinal pigment epithelium layer boundary 105 has distortion, the process advances to step S1365.

In step S1365, the part determination unit 1256 determines whether the part determined in step S1330 is the optic disc portion. Upon determining in step S1365 that the part is the optic disc portion, the process advances to step S1370.

If the part is the macular portion, the image processing apparatus 1201 executes, in step S1370, an image analysis algorithm (normal macular portion feature processing) for a case in which neither the cyst 107 nor the white spot 108 exists, and the retinal pigment epithelium layer boundary 105 has no distortion (when the macular portion is normal). In other words, the normal macular portion feature processing is processing of calculating diagnosis information data effective for quantitatively diagnosing the presence/absence of glaucoma, the degree of progress of glaucoma, and the like in the macular portion. Note that details of the normal macular portion feature processing are fundamentally the same as those of the normal eye feature processing described in the first embodiment with reference to FIG. 6, and a description thereof will not be repeated here.

In the normal eye feature processing shown in FIG. 6, processing of acquiring or detecting the inner limiting membrane 103, the nerve fiber layer boundary 104 or an outer boundary 104a of inner plexiform layer, and the retinal pigment epithelium layer boundary 105 is executed in step S620. In the normal macular portion feature processing (step S1370), however, processing of acquiring or detecting the inner limiting membrane 103, the nerve fiber layer boundary 104 or the outer boundary 104a of inner plexiform layer, and the retinal pigment epithelium layer boundary 105 included in the search range 1004 in FIG. 10 is performed.

If the part is the optic disc portion, the image processing apparatus executes, in step S1370, an image analysis algorithm (abnormal optic disc portion feature processing) for a case in which neither the cyst 107 nor the white spot 108 exists, and the retinal pigment epithelium layer boundary 105 has distortion (when the optic disc portion is abnormal). In other words, the abnormal optic disc portion feature processing is processing of calculating diagnosis information data effective for quantitatively diagnosing the shape of the depressed portion of the optic disc portion.

Note that the abnormal optic disc portion feature processing is fundamentally the same as the normal eye feature processing described in the first embodiment with reference to FIG. 6, and a detailed description thereof will not be repeated here. In the normal eye feature processing shown in FIG. 6, the quantification unit 462 performs, in step S630, processing of quantifying the thickness of the nerve fiber layer 102 and the thickness of entire retinal layer based on the nerve fiber layer boundary 104 acquired in step S620. In the abnormal optic disc portion feature processing, however, not the processing of quantifying the thicknesses but processing of quantifying an indicator representing the shape of the depressed portion 1101 or 1102 of the optic disc portion shown in 11a or 11b of FIG. 11 (processing of calculating the area or volume of the depressed portion) is performed.

On the other hand, if the state determination unit 453 determines in step S1355 that the eye features include the white spot 108 or cyst 107, or if the part determination unit 1256 determines in step S1365 that the part is the macular portion, the process advances to step S1375.

In step S1375, an image processing unit 430 executes an image analysis algorithm (abnormal macular portion feature processing) for a case in which it is determined that the macular portion includes, as an eye feature, the cyst 107, white spot 108, or distortion of the retinal pigment epithelium layer boundary 105. Note that the abnormal macular portion feature processing is fundamentally the same as the abnormal eye feature processing described in the first embodiment with reference to FIGS. 7, 8A, and 8B, and a description thereof will not be repeated here.

In the processing for age-rated macular degeneration shown in FIG. 8B, the processing target change unit 454 instructs the layer decision unit 461 in step S815 to newly detect the normal structure 106 of the retinal pigment epithelium layer boundary as a detection target. In the abnormal macular portion feature processing, however, a layer decision unit 461 is instructed to detect a normal structure 106 within the search range 1004 in FIG. 10.

As is apparent from the above description, the image processing apparatus according to the embodiment is configured to determine a part in an acquired wide-angle tomogram, and change a detection target to be detected or detection parameters for detection for each determined part in accordance with the eye state.

This makes it possible to accurately acquire diagnosis information parameters effective for diagnosing the presence/absence of various kinds of diseases such as glaucoma, age-rated macular degeneration, and macular edema and the degree of progress of the diseases even in a wide-angle tomogram.

### Third Embodiment

In the above-described first and second embodiments, the apparatus is configured to calculate, as diagnosis information data, the nerve fiber layer thickness, the thickness of entire retinal layer, the area (volume) of a region between the actual measured position and the estimated position of retinal pigment epithelium layer boundary, and the like. However, the present invention is not limited to this. For example, the apparatus may be configured to obtain diagnosis information data from tomograms of different imaging dates/times (imaging timings), compare them with each other to quantify the time-rate change, and output new diagnosis information data (follow-up diagnosis information data). More specifically, two tomograms of different imaging dates/times are aligned based on a predetermined alignment target included in each tomogram, and the difference between corresponding diagnosis information data is obtained, thereby quantifying the time-rate change between the two tomograms. Note that in the following description, a tomogram to be aligned will be referred to as a reference image (first tomogram), and a tomogram to be deformed and moved for alignment will be referred to as a floating image (second tomogram).

Note that in this embodiment, image analysis processing described in the first embodiment is executed for both the reference image and the floating image, and calculated diagnosis information data are already stored in a data server 202.

This embodiment will be described below in detail. Note that the overall arrangement of the diagnostic imaging system and the hardware configuration of the image processing apparatus are the same as in the first embodiment, and a description thereof will not be repeated here.

<1. Relationship between Eye States, Eye Features, Alignment Targets, and Follow-Up Diagnosis Information Data>

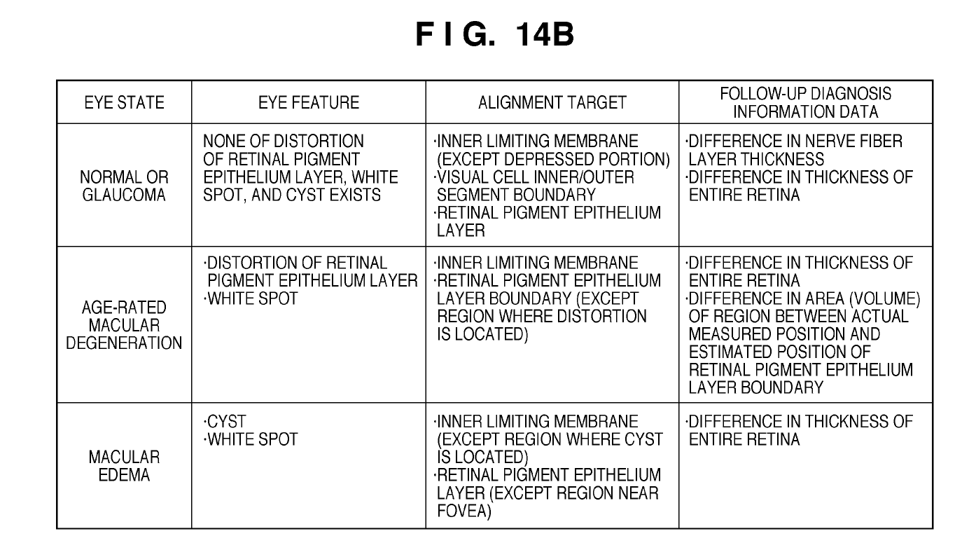

The relationship between eye states, eye features, alignment targets, and follow-up diagnosis information data will be explained first. In FIG. 14A, 14a to 14f are schematic views of two tomograms of retina captured by an OCT. When aligning tomograms of different imaging dates/times (imaging timings), the image processing apparatus according to the embodiment selects a hard-to-deform region as an alignment target for each eye state. Then, alignment processing corresponding to the eye state (alignment processing using an optimized coordinate transformation method, alignment parameters, and weight of alignment similarity calculation) is executed for the floating image using the selected alignment target.

In FIG. 14A, 14a and 14b are schematic views of tomograms of the optic disc portion of retina captured by an OCT (enlarged views of an inner limiting membrane 103). Referring to 14a and 14b in FIG. 14A, reference numeral 1401 or 1402 denotes a depressed portion of the optic disc portion. In general, a nerve fiber layer 102 or the inner limiting membrane 103 near the depressed portion of the optic disc portion is a region that readily deforms. For this reason, when aligning tomograms including the optic disc portion, the inner limiting membrane 103 except the depressed portion of the optic disc portion, visual cell inner/outer segment boundary (IS/OS), and the retinal pigment epithelium layer boundary are selected as alignment targets (bold line portions in 14a and 14b of FIG. 14A).

In FIG. 14A, 14c and 14d show tomograms of retina of a patient suffering from macular edema. In macular edema, hard-to-deform regions are the inner limiting membrane 103 except the region where a cyst 107 is located, and a retinal pigment epithelium layer boundary 105 except a region near the fovea (bold line portions in 14c and 14d of FIG. 14A). For this reason, when aligning tomograms determined to include macular edema, the inner limiting membrane 103 except the region where the cyst 107 is located and the retinal pigment epithelium layer boundary 105 except the region near the fovea are selected as alignment targets.

In FIG. 14A, 14e and 14f show tomograms of retina of a patient suffering from age-rated macular degeneration. In age-rated macular degeneration, hard-to-deform regions are the inner limiting membrane 103 and the retinal pigment epithelium layer boundary 105 except a region where distortion is located (bold line portions in 14e and 14f of FIG. 14A). For this reason, when aligning tomograms determined to include age-rated macular degeneration, the inner limiting membrane 103 and the retinal pigment epithelium layer boundary 105 except the region where distortion is located are selected as alignment targets.

Note that the alignment targets are not limited to those. When a normal structure 106 of the retinal pigment epithelium layer boundary is calculated, the normal structure of the retinal pigment epithelium layer boundary may be selected as an alignment target (bold dotted line portions in 14e and 14f of FIG. 14A).

In FIG. 14B is a table that provides a summary of the relationship between eye states, eye features, alignment targets, and follow-up diagnosis information data. An image processing apparatus according to the embodiment which executes image analysis processing based on the table shown in FIG. 14B will be described below in detail.

<2. Functional Arrangement of Image Processing Apparatus>

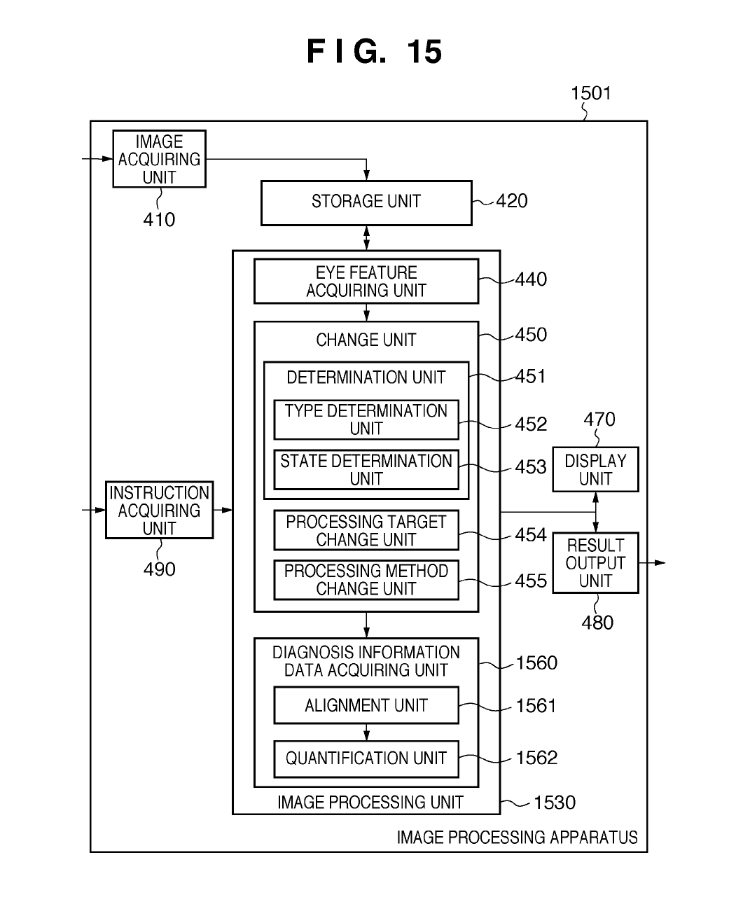

The functional arrangement of an image processing apparatus 1501 according to the embodiment will be described first with reference to FIG. 15. FIG. 15 is a block diagram showing the functional arrangement of the image processing apparatus 1501 according to the embodiment. This apparatus is different from the image processing apparatus 201 (FIG. 4) according to the first embodiment in that an alignment unit 1561 is arranged in a diagnosis information data acquiring unit 1560 in place of the layer decision unit 461. In addition, a quantification unit 1562 calculates follow-up diagnosis information data obtained by quantifying the time-rate change between two tomograms aligned by the alignment unit 1561. The functions of the alignment unit 1561 and the quantification unit 1562 will be described below.

(1) Functions of Alignment Unit 1561

The alignment unit 1561 selects alignment targets based on an instruction from a processing target change unit 454 (in this case, an instruction about alignment targets corresponding to the eye state). The alignment unit 1561 also executes alignment processing (alignment processing using an optimized coordinate transformation method, alignment parameters, and weight of alignment similarity calculation) based on an instruction from a processing method change unit 455 (in this case, an instruction about alignment processing corresponding to the eye state). This is because when aligning tomograms of different imaging dates/times for follow-up, the type and range of layer or tissue that readily deforms changes depending on the eye state.

More specifically, if a state determination unit 453 determines that none of distortion of the retinal pigment epithelium layer boundary 105, white spot 108, and cyst 107 is included, the inner limiting membrane 103 except the depressed portion of the optic disc portion is selected as an alignment target. The visual cell inner/outer segment boundary (IS/OS) and the retinal pigment epithelium layer boundary are also selected.

When none of distortion of the retinal pigment epithelium layer boundary 105, white spot 108, and cyst 107 is included, deformation of retina is relatively small, and therefore, a rigid-body transformation method is selected as the coordinate transformation method. As the alignment parameters, translation (x,y,z) and rotation (α,β,γ) are selected. However, the coordinate transformation method is not limited to this, and for example, an Affine transformation method or the like may be selected. Furthermore, the weight of alignment similarity calculation is set to be small in a region (false image region) under the retinal blood vessel region (in a region deeper than blood vessels of retina).

The weight of alignment similarity calculation is set to be small in the false image region under the retinal blood vessel region due to the following reason.

Generally, the region deeper than the blood vessels of retina includes a region (false image region) with an attenuated luminance value. The position (direction) the false image region is generated changes depending on the irradiation direction of light source. For this reason, the false image region generation position may change due to the difference in imaging condition between the reference image and the floating image. Hence, it is effective to set the weight of alignment similarity calculation to be smaller in the false image region. Note that setting the weight to 0 is equivalent to excluding the region from the alignment similarly processing target.

On the other hand, when the state determination unit 453 determines that the cyst 107 is included, the alignment unit 1561 selects, as alignment targets, the inner limiting membrane 103 and the retinal pigment epithelium layer boundary 105 except a region near the fovea (bold line portions in 14c and 14d of FIG. 14A).

In this case, rigid-body transformation is selected as the coordinate transformation method. As the alignment parameters, translation (x,y,z) and rotation (α,β,γ) are selected. However, the coordinate transformation method is not limited to this, and for example, an Affine transformation method or the like may be selected. Furthermore, the weight of alignment similarity calculation is set to be small in the false image region under the retinal blood vessel region and a white spot region. First alignment processing is performed under these conditions.

After the first alignment processing, FFD (Free From Deformation) that is a kind of non-rigid body transformation is selected as the coordinate transformation method, and second alignment processing is performed. Note that in FFD, each of the reference image and the floating image is divided into local blocks, and block matching is performed between the local blocks. For a local block including an alignment target, the search range for block matching is set to be narrower than that in the first alignment processing.

If the state determination unit 453 determines that the white spot 108 and distortion of the retinal pigment epithelium layer boundary are included, the alignment unit 1561 selects, as alignment targets, the inner limiting membrane 103 and the retinal pigment epithelium layer boundary 105 except the region where distortion is detected. More specifically, the bold line portions in 14e and 14f of FIG. 14A are selected. However, the alignment targets are not limited to those. For example, the normal structure 106 of the retinal pigment epithelium layer boundary may be obtained in advance and selected (bold dotted line portions in 14e and 14f of FIG. 14A).

Upon determining that the white spot 108 and distortion of the retinal pigment epithelium layer boundary are included, rigid-body transformation is selected as the coordinate transformation method. As the alignment parameters, translation (x,y,z) and rotation (α,β,γ) are selected. However, the coordinate transformation method is not limited to this, and for example, an Affine transformation method or the like may be selected. Furthermore, the weight of alignment similarity calculation is set to be small in the false image region under the retinal blood vessel region and a white spot region. First alignment processing is performed under these conditions. After the first alignment processing, FFD is selected as the coordinate transformation method, and second alignment processing is performed. Note that in FFD, each of the reference image and the floating image is divided into local blocks, and block matching is performed between the local blocks.

(2) Functions of Quantification Unit 1562

The quantification unit 1562 calculates follow-up diagnosis information parameters by quantifying the time-rate change between the two tomograms based on the tomograms that have undergone the alignment processing. More specifically, diagnosis information data for the reference image and the floating image are read out from the data server 202. The diagnosis information data for the floating image is processed based on the alignment processing result (alignment evaluation value), and compared with the diagnosis information data for the reference image. This allows to calculate the differences in the nerve fiber layer thickness, thickness of entire retinal layer, and area (volume) of a region between the actual measured position and the estimated position of retinal pigment epithelium layer boundary (that is, the quantification unit 1562 functions as a difference calculation unit).

<3. Procedure of Image Analysis Processing of Image Processing Apparatus>

The procedure of image analysis processing of the image processing apparatus 1501 will be described next. Note that the procedure of image analysis processing of the image processing apparatus 1501 is fundamentally the same as image analysis processing of the image processing apparatus 201 according to the first embodiment (FIG. 5). However, the processing is different from image analysis processing of the image processing apparatus 201 according to the first embodiment (FIG. 5) in normal eye feature processing (step S560) and abnormal eye feature processing (step S565). Normal eye feature processing (step S560) and abnormal eye feature processing (step S565) will be explained below in detail. Note that in the detailed abnormal eye feature processing (S565) shown in FIG. 7, only processing for macular edema (step S720) and processing for age-rated macular degeneration (step S725) are different, and these processes will be described below.

<Procedure of Normal Eye Feature Processing>

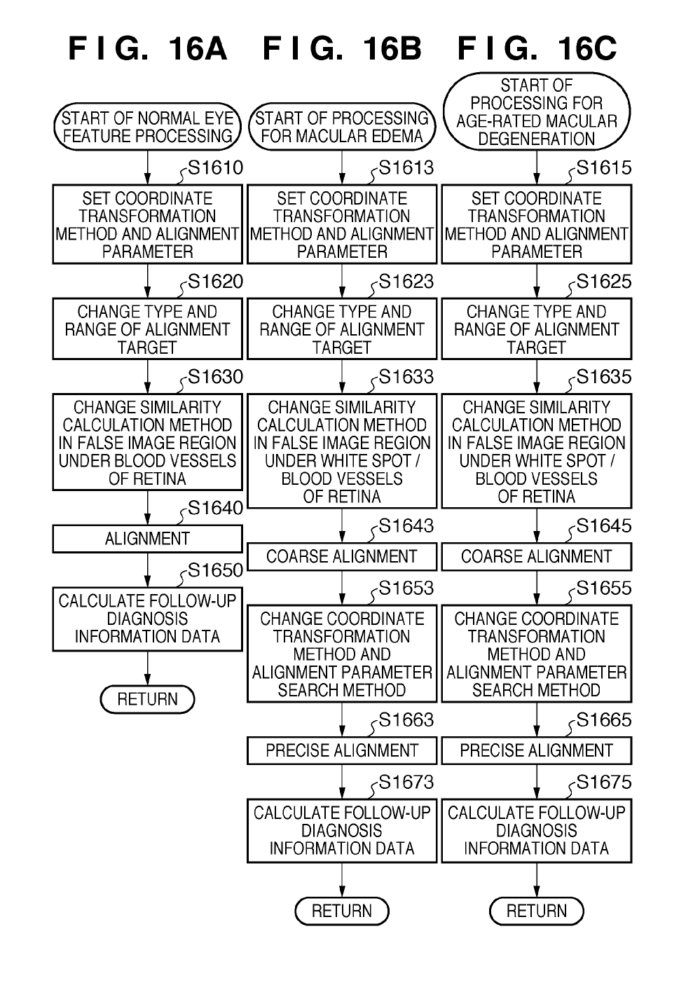

FIG. 16A is a flowchart illustrating the procedure of normal eye feature processing of the image processing apparatus 1501 according to the embodiment.

In step S1610, the alignment unit 1561 sets the coordinate transformation method and alignment parameters. Note that in the normal eye feature processing executed upon determining that none of distortion of the retinal pigment epithelium layer boundary, white spot, and cyst exists, the image analysis target is the floating image including relatively small retinal deformation, and therefore, the rigid-body transformation method is selected as the coordinate transformation method. As the alignment parameters, translation (x,y,z) and rotation (α,β,γ) are selected.

In step S1620, the alignment unit 1561 selects, as alignment targets, the inner limiting membrane 103 except the depressed portion of the optic disc portion, visual cell inner/outer segment boundary (IS/OS), and RPE (Retinal Pigment Epithelium) layer.

In step S1630, the weight of alignment similarity calculation in the false image region under the retinal blood vessel region is set to be small. More specifically, the weight of alignment similarity calculation is set to a value from 0 (inclusive) to 1.0 (exclusive) in a range defined by the OR of regions each having the same x- and y-coordinates as those of a blood vessel of retina and a z-coordinate value larger than that of the inner limiting membrane 103 on the reference image and the floating image.

In step S1640, the alignment unit 1561 performs alignment processing using the coordinate transformation method, alignment parameters, alignment targets, and weight set in steps S1610, S1620, and S1630, and obtains an alignment evaluation value.

In step S1650, the quantification unit 1562 acquires diagnosis information data for the floating image and that for the reference image from the data server 202. The diagnosis information data for the floating image is processed based on the alignment evaluation value, and compared with the diagnosis information data for the reference image, thereby quantifying the time-rate change between them and outputting follow-up diagnosis information data. More specifically, the difference in the thickness of entire retina is output as follow-up diagnosis information data.

<Procedure of Processing for Macular Edema>

The procedure of processing for macular edema will be described next in detail with reference to FIG. 16B. In step S1613, the alignment unit 1561 sets the coordinate transformation method and alignment parameters. More specifically, the alignment unit 1561 selects the rigid-body transformation method as the coordinate transformation method, and translation (x,y,z) and rotation (α,β,γ) as the alignment parameters.

In step S1623, the alignment unit 1561 changes the alignment targets. When the cyst 107 is extracted as an eye feature (when the eye state is determined as macular edema), the retinal pigment epithelium layer boundary deforms near the fovea of the macular portion at a high probability. The visual cell inner/outer segment boundary (IS/OS) may disappear along with the progress of disease. Hence, the inner limiting membrane 103 and the retinal pigment epithelium layer boundary 105 except the region near the fovea (bold line portions in 14c and 14d of FIG. 14A) are selected as alignment targets.

In step S1633, the alignment unit 1561 sets the weight of alignment similarity calculation to be small in the false image region under the regions of the blood vessels of retina and the white spot 108. Note that the similarity calculation method for the false image region under the retinal blood vessel region is the same as in step S1630, and a description thereof will not be repeated here.

More specifically, the weight of alignment similarity calculation is set to a value from 0 (inclusive) to 1.0 (exclusive) in a range defined by the OR of the following regions:

a region having the same x- and y-coordinates as those of the white spot 108 and a z-coordinate value larger than that of the white spot 108 on the reference image; and

a region having the same x- and y-coordinates as those of the white spot 108 and a z-coordinate value larger than that of the white spot 108 on the floating image.

In step S1643, the alignment unit 1561 performs coarse alignment (first alignment processing) using the coordinate transformation method, alignment parameters, alignment targets, and weight set in steps S1613 to S1633. The alignment unit 1561 also obtains an alignment evaluation value.

In step S1653, the alignment unit 1561 changes the coordinate transformation method and the search range of alignment parameters for precise alignment (second alignment processing).

In this case, the coordinate transformation method is changed to FFD (Free From Deformation) that is a kind of non-rigid body transformation. The search range of alignment parameters is set to be narrower. Note that in FFD, each of the reference image and the floating image is divided into local blocks, and block matching is performed between the local blocks. On the other hand, for macular edema, the type and range of a hard-to-deform layer serving as a mark for alignment are indicated by the bold line portions in 14c and 14d of FIGS. 14. Hence, when executing FFD, the search range for block matching is set to be narrower for local blocks including the bold line portions in 14c and 14d of FIG. 14A.

In step S1663, the alignment unit 1561 performs precise alignment based on the coordinate transformation method and alignment parameter search range set in step S1633, and obtains an alignment evaluation value.

In step S1673, the quantification unit 1562 acquires diagnosis information data for the floating image and that for the reference image from the data server 202. The diagnosis information data for the floating image is processed based on the alignment evaluation value, and compared with the diagnosis information data for the reference image, thereby quantifying the time-rate change between them and outputting follow-up diagnosis information data. More specifically, the difference in the thickness of entire retina near the fovea is output as follow-up diagnosis information data.

<Procedure of Processing for Age-Rated Macular Degeneration>

The procedure of processing for age-rated macular degeneration will be described next in detail with reference to FIG. 16C. In step S1615, the alignment unit 1561 sets the coordinate transformation method and alignment parameters. More specifically, the alignment unit 1561 selects the rigid-body transformation method as the coordinate transformation method, and translation (x,y,z) and rotation (α,β,γ) as the alignment parameters.

In step S1625, the alignment unit 1561 changes the alignment targets. When distortion of the retinal pigment epithelium layer is extracted as an eye feature (when the eye state is determined as age-rated macular degeneration), the range in which distortion of the retinal pigment epithelium layer is extracted and its neighboring region readily deform. The visual cell inner/outer segment boundary (IS/OS) may disappear along with the progress of disease. Hence, the inner limiting membrane 103 and the retinal pigment epithelium layer boundary 105 except the region where distortion is extracted (bold line portions in 14e and 14f of FIGS. 14) are selected as alignment targets. Note that the alignment targets are not limited to those. For example, the normal structure 106 of the retinal pigment epithelium layer boundary (bold dotted line portions in 14e and 14f of FIG. 14A) may be obtained in advance and selected.

In step S1635, the alignment unit 1561 sets the weight of alignment similarity calculation to be small in the false image region under the regions of the blood vessels of retina and the white spot 108. Note that the similarity calculation processing is the same as that of step S1633, and a detailed description thereof will not be repeated here.

In step S1645, the alignment unit 1561 performs coarse alignment (first alignment processing) using the coordinate transformation method, alignment parameters, alignment targets, and weight set in steps S1615 to S1635. The alignment unit 1561 also obtains an alignment evaluation value.

In step S1655, the alignment unit 1561 changes the coordinate transformation method and the search method in the alignment parameter space for precise alignment (second alignment processing).

As in step S1653, the coordinate transformation method is changed to FFD, and the search range of alignment parameters is set to be narrower. Note that for age-rated macular degeneration, the type and range of hard-to-deform layer serving as a mark for alignment are indicated by the bold line portions in 14e and 14f of FIG. 14A. Hence, the search range for block matching is set to be narrower for local blocks including the bold line portions.

In step S1665, the alignment unit 1561 performs precise alignment based on the coordinate transformation method and alignment parameter search range set in step S1655, and obtains an alignment evaluation value.

In step S1675, diagnosis information data for the floating image and that for the reference image are acquired from the data server 202. The diagnosis information data for the floating image is processed based on the alignment evaluation value, and compared with the diagnosis information data for the reference image, thereby quantifying the time-rate change between them and outputting follow-up diagnosis information data. More specifically, the difference in the area (volume) of a region corresponding to the blood vessels of retina, that is, a region between the actual measured position and the estimated position of retinal pigment epithelium layer boundary is output as follow-up diagnosis information data.

As is apparent from the above description, the image processing apparatus according to the embodiment is configured to align tomograms of different imaging dates/times using alignment targets corresponding to the eye state to quantify the time-rate change between them.

Executing an image analysis algorithm corresponding to the eye state makes it possible to accurately calculate, independently of the eye state, follow-up diagnosis information parameters effective for diagnosing the degree of progress of various kinds of diseases such as glaucoma, age-rated macular degeneration, and macular edema.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-278948 filed Dec. 8, 2009, which is hereby incorporated by reference herein in its entirety.

What is claimed is:

1. An image processing apparatus for processing a tomogram of an eye, comprising:
a determination unit configured to determine a state of a disease in the eye based on information of the tomogram; and
a detection unit configured to change, in accordance with the state of the disease in the eye determined by said determination unit, one of a detection target to be used to calculate diagnosis information data quantitatively representing the state of the disease and an algorithm to be used to detect the detection target.

2. The apparatus according to claim 1, wherein
the detection target includes a predetermined layer of the tomogram, and
if a shape of the predetermined layer has changed, or the tomogram includes a predetermined tissue, said detection unit changes a detection parameter to be used to detect the predetermined layer included in the detection target, and then redetects the predetermined layer.

3. The apparatus according to claim 2, wherein presence/absence of the change of the shape of the predetermined layer includes presence/absence of distortion of retinal pigment epithelium layer of the eye, and presence/absence of the predetermined tissue includes one of presence/absence of a white spot and presence/absence of a cyst.

4. The apparatus according to claim 3, wherein
said determination unit determines
a first state upon determining that the distortion of the retinal pigment epithelium layer of the eye does not exist, and neither the white spot nor the cyst exists,
a second state upon determining that the distortion of the retinal pigment epithelium layer of the eye exists, or not the cyst but the white spot exists, and
a third state upon determining the cyst exists, and
said detection unit detects
an inner limiting membrane, a nerve fiber layer boundary, and a retinal pigment epithelium layer boundary as the detection target when said determination unit has determined the first state,
the inner limiting membrane, the retinal pigment epithelium layer boundary, and a retinal pigment epithelium layer boundary assuming that the retinal pigment epithelium layer has no distortion as the detection target when said determination unit has determined the second state, and
the inner limiting membrane and the retinal pigment epithelium layer boundary as the detection target when said determination unit has determined the third state.

5. The apparatus according to claim 4, wherein
when said determination unit has determined that the distortion of the retinal pigment epithelium layer of the eye exists, said detection unit changes a detection parameter to be used to detect the retinal pigment epithelium layer boundary in a region with the distortion, and then redetects the retinal pigment epithelium layer boundary,
when said determination unit has determined that the white spot exists, said detection unit changes a detection parameter to be used to detect the retinal pigment epithelium layer boundary located at a deep position in a depth direction relative to the determined white spot, and then redetects the retinal pigment epithelium layer boundary.

6. The apparatus according to claim 4, further comprising a calculation unit configured to calculate
a nerve fiber layer thickness and a thickness of entire retina as the diagnosis information data when said determination unit has determined the first state,
the thickness of entire retina and an area or volume of a region between the retinal pigment epithelium layer boundary and the retinal pigment epithelium layer boundary assuming that the retinal pigment epithelium layer has no distortion as the diagnosis information data when said determination unit has determined the second state, and
the thickness of entire retina as the diagnosis information data when said determination unit has determined the third state.

7. The apparatus according to claim 4, further comprising a specifying unit configured to extract a depressed portion of the inner limiting membrane so as to extract an optic disc portion and a macular portion of the eye, and specify the optic disc portion and the macular portion based on presence/absence of blood vessels of retina and a nerve fiber layer thickness in the depressed portion,
wherein the tomogram of the eye undergoes processing for each part specified by said specifying unit.

8. The apparatus according to claim 6, further comprising:
an alignment unit configured to align a first tomogram whose diagnosis information data is calculated by said calculation unit with a second tomogram whose diagnosis information data is calculated by said calculation unit and whose imaging timing is different from that of the first tomogram; and
a difference calculation unit configured to calculate follow-up diagnosis information data representing a difference between the diagnosis information data of the first tomogram and that of the second tomogram by obtaining a difference in specified position information between the first tomogram and the second tomogram which are aligned by said alignment unit.

9. The apparatus according to claim 8, wherein said alignment unit
performs alignment based on, out of the detection target detected by said detection unit, a region selected as a reference in accordance with the state of the disease of the eye determined by said determination unit, and
performs alignment using a processing method selected in accordance with the state of the disease of the eye determined by said determination unit.

10. An image processing method of an image processing apparatus for processing a tomogram of an eye, comprising:
causing a determination unit to determine a state of a disease in the eye based on information of the tomogram; and
causing a detection unit to change, in accordance with the state of the disease in the eye determined by the determination unit, one of a detection target to be used to calculate diagnosis information data quantitatively representing the state of the disease and an algorithm to be used to detect the detection target.

11. A computer-readable storage medium storing a program that causes a computer to execute steps of an image processing method of claim 10.
